(19) United States
(12) Patent Application Publication
(10) Pub. No.: US 2025/0025092 A1
(43) Pub. Date: Jan. 23, 2025

(54) NEURO-PHYSIOLOGICAL REHABILITATION SYSTEM AND METHOD

(71) Applicant: CLR Neurosthenics, Inc., Manhattan Beach, CA (US)

(72) Inventors: Michael D. MARK, Los Angeles, CA (US); Mark Charles O'BRYAN, Dana Point, CA (US); Audrey HILL-LINDSAY, Huntington Beach, CA (US)

(21) Appl. No.: 18/709,464

(22) PCT Filed: Nov. 14, 2022
(86) PCT No.: PCT/US2022/049852

§ 371 Date: May 10, 2024

Related U.S. Application Data
(60) Provisional application No. 63/279,107, filed on Nov. 13, 2021.

(51) Int. Cl. A61B 5/378; A61B 5/00; A61B 5/0205; A61B 5/16; A61B 5/375

(52) U.S. Cl. CPC A61B 5/378; A61B 5/02055; A61B 5/165; A61B 5/375; A61B 5/743; A61B 2505/09

(57) ABSTRACT

The disclosed system provides a neuro-physiological assessment and rehabilitation system. The system selects, based on an injury type, one or more target regions of the patient's brain for assessment of a neurological metric associated with a neurological network of the patient's brain and a first predetermined visual stimulation is provided to the patient while first neurological data pertaining to the athletic performance of the patient reacting to the visual stimulation is received, including measurements pertaining to the neurological metric. The obtained first neurological data is compared to the baseline data threshold and a second visual stimulation configured to prompt the neurological metric toward the baseline data threshold is selected. Second neurological data pertaining to the athletic performance of the patient reacting to the second visual stimulation is received and a state of the neurological metric indicating whether the neurological metric adjusted toward the baseline threshold is provided.

### CROSS REFERENCE TO RELATED APPLICATIONS

This application claims the benefit of U.S. Provisional Application No. 63/279,107 filed on Nov. 13, 2021, and entitled NEURO-PHYSIOLOGICAL REHABILITATION SYSTEM AND METHOD, the entirety of which is incorporated herein by reference for all purposes.

### BACKGROUND FIELD

The subject technology addresses deficiencies commonly encountered in the assessment and rehabilitation of athletes after injury.

### SUMMARY

Somatic rehabilitation has been explored for post lower extremity injuries. However, current rehab methods and return to activity decisions lack sufficient examination of brain and physiometric rehabilitation parameters with regard to prevention of re-injury.

The subject technology provides a multimodal, neuroplasticity assessment and rehabilitation training tool that provides training protocols for facilitating injury rehabilitation and prevention, and to assess and maximize athletic performance. The disclosed system further facilitates optimization of rehabilitation throughout the athletic lifecycle. The system collects physiological and neurological data from an athlete during an assessment program. The collected data is analyzed to identify the impact of the athlete's mental status (e.g., state of mind) on the athlete's physical capabilities. Similarly, the disclosed system and method identifies the impact of the athlete's physical capabilities on the athlete's mental capabilities. The subject technology utilizes, among other data collection technologies, qEEG to improve rehabilitation injury management, thereby providing a tool for more informed decision making with respect to when injured athletes are able to return to play. Based on assessment data, the disclosed system automatically determines and assigns physical and mental tasks to the athlete to rehabilitate physiological and neurological functions to facilitate recovery of physical injury.

According to some implementations, the subject technology includes a method for physiological and neurological rehabilitation of an athlete. According to various aspects, the method includes obtaining first physiological and neurological data pertaining to athletic performance of a person reacting to predetermined visual (or audible) stimulation: determine a baseline data threshold for the obtained first physiological and neurological data; selecting second visual stimulation based on the obtained physiological and neurological data and the baseline data threshold; obtaining, after the second visual stimulation is selected, second physiological and neurological data pertaining to one or more actions of the person responsive to the altered visual stimulation: determining a deviation between the baseline data threshold and the second physiological and neurological data; and generating a neurological and physiological protocol based on the deviation. Other aspects include corresponding systems, apparatuses, and computer program products for implementation of the computer-implemented method.

In some implementations, a method includes receiving an injury type pertaining to a physical injury experienced by a patient; selecting, based on an injury type, one or more target regions of the patient's brain for assessment of a neurological metric associated with a neurological network of the patient's brain; providing one or more neurological sensors for measuring neurological activity within the one or more target regions of the patient's brain; providing, via a display screen, a first predetermined visual stimulation; initiating an athletic performance of the patient based on the first predetermined visual stimulation; obtaining, via one or more neurological sensors, first neurological data pertaining to the athletic performance of the patient reacting to the first predetermined visual stimulation, wherein the first neurological data comprises measurements pertaining to the neurological metric; determine a baseline data threshold for the neurological metric; comparing the obtained first neurological data to the baseline data threshold; selecting a second predetermined visual stimulation configured to prompt the neurological metric toward the baseline data threshold; obtaining, via the one or more neurological sensors, during the second predetermined visual stimulation, second neurological data pertaining to one or more actions of the patient responsive to the second predetermined visual stimulation; determining a deviation between the baseline data threshold and the second neurological data; determining, based on the deviation, a state of the neurological metric indicating whether the neurological metric adjusted toward the baseline threshold; and providing, on a display screen, an indication of the state of the neurological metric and a graphical representation of the second neurological data and the deviation. Other aspects include corresponding systems, apparatuses, and computer program products for implementation of the computer-implemented method.

Further aspects of the subject technology, features, and advantages, as well as the structure and operation of various aspects of the subject technology are described in detail below with reference to accompanying drawings.

### DESCRIPTION OF THE FIGURES

Various objects, features, and advantages of the present disclosure can be more fully appreciated with reference to the following detailed description when considered in connection with the following drawings, in which like reference numerals identify like elements. The following drawings are for the purpose of illustration only and are not intended to be limiting of this disclosure, the scope of which is set forth in the claims that follow.

FIG. 1 is a conceptual diagram illustrating an example system for rehabilitation of an athlete, according to various aspects of the subject technology.

FIGS. 2A, 2B, and 2C depict an example biometric interface for initiating and controlling patient physiological and neurological, according to various aspects of the subject technology.

FIG. 3 depicts an example process for physiological and neurological rehabilitation of an athlete, according to aspects of the subject technology.

FIG. 4 depicts example physical and mental tasks utilized by the subject technology to induce and measure a neuro-physical response, according to aspects of the subject technology.

FIGS. 5A through 5Z depict example reports and associated metrics provided by the disclosed system.

FIGS. 6A through 6B depict example neurological data assessments and visualizations that may be produced by the biometric interface.

FIG. 7 depicts example physiological data assessments and visualizations that may be produced by the biometric interface.

FIG. 8 is a conceptual system diagram illustrating an example software system, including a database, for physiological and neurological rehabilitation of an athlete, according to aspects of the subject technology.

FIG. 9 is a conceptual diagram illustrating an example electronic system for physiological and neurological rehabilitation of an athlete, according to aspects of the subject technology.

### DESCRIPTION

While aspects of the subject technology are described herein with reference to illustrative examples for particular applications, it should be understood that the subject technology is not limited to those particular applications. Those skilled in the art with access to the teachings provided herein will recognize additional modifications, applications, and aspects within the scope thereof and additional fields in which the subject technology would be of significant utility.

The subject technology addresses various challenges related to prescribing patient rehabilitation that exist in clinical environments where many different data sources are available to make rehabilitation decisions. Multiple devices may contribute to a patient's care, particularly when focused on the rehabilitation of athletes. For example, the clinicians may rely on a substantial amount of lab data in the form of, for example, patient electronic medical records, test results, etc., as well as subjective determinations of athletic performance to put together a story about an athlete to determine a protocol for rehabilitation. Each type of data and/or system may be viewed in isolation in one particular area without holistically capturing an accurate picture of an athlete's performance and progress toward rehabilitative goals, particularly after injury.

Whereas some methods may identify neuro-deficient brain activity and monitor improvement in neuro-deficient activity over time as a patient is rehabilitated, such methods of rehabilitation are focused on exercises that are independent of neurological rehabilitation. Contrary to such methods, the subject technology addresses existing rehabilitation deficiencies by providing a comprehensive real-time analysis system that focuses rehabilitation on neurological and physiological functions to rehabilitate physical injury, thereby providing for more informed decision making with respect to determining when injured athletes are able to return to play.

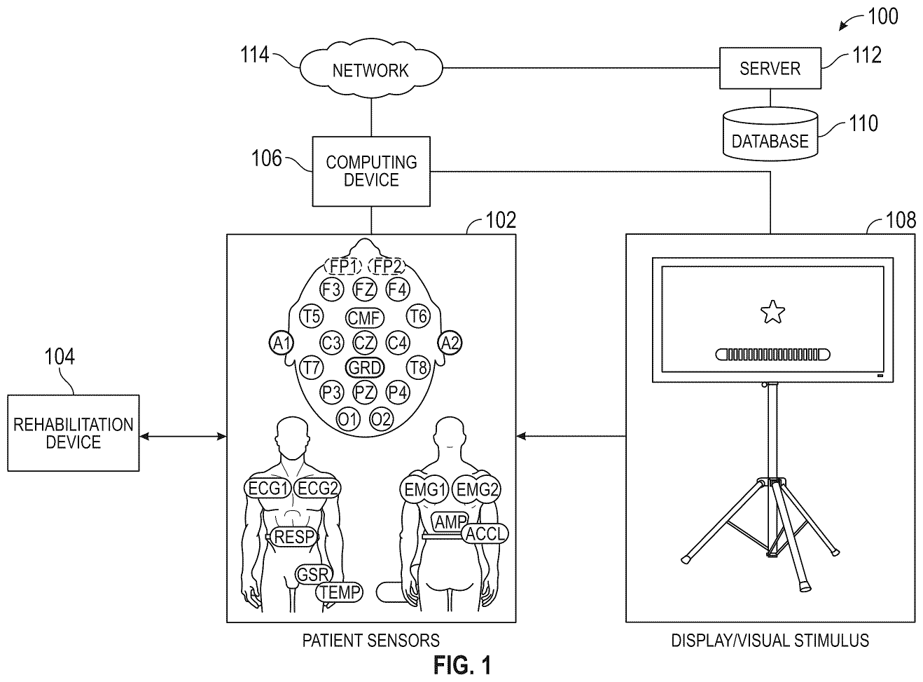

FIG. 1 is a conceptual diagram illustrating an example system 100 for rehabilitation of an athlete, according to various aspects of the subject technology. System 100 includes (among other equipment) one or more neurological and/or physiological sensor devices 102, one or more rehabilitation devices 104, a computing device 106, a stimulus device 108, and a data source repository or database 110. Database 110 may be directly or operably connected to computing device 106 via a local wired or wireless connection, or may be connected to a server 112 remote from computing device 106, which may be connected to computing device 106 via a network 114. The centralized server 112 and data source(s) 110 may include multiple computing devices distributed over a local or wide area network 114, or may be combined in a single device. Patient data may be stored in data source(s) 110 in real time and accessed and/or managed by a centralized server 112 and/or computing device 106. In this regard, multiple devices (including, e.g., sensors 102, rehabilitation devices 104 and/or computing device 108) may communicate patient data, over network 114, to centralized server 112 in real time as the data is collected or measured from the patient, and centralized server 112 may store the patient data in data source(s) 110. According to some implementations, one or more servers may receive and store the patient data in multiple data sources.

According to various implementations, computing device 106 is configured (by way of instructions) to generate and provide a virtual interface and/or stimulus, to stimulus device 108, to prompt the patient to take certain actions, either independently or in conjunction with operation of the rehabilitation device 104. In some implementations, stimulus device 108 may include a display screen that renders the interface for presentation to the athlete. In some implementations, stimulus device 108 may include a virtual reality (VR) headset or glasses or an augmented reality device worn by the athlete. In some implementations, the stimulus device 108 may be integrated in computing device 106, so as to provide instructions and/or prompts for guiding the patient to take certain actions.

The depicted computing device 106 may include personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity. The stimulus device 108 may include an auditory device, such as headphones or speaker system configured to provide audio prompts to the athlete. In some implementations, stimulus device 108 may include haptic or tactile feedback, or may include a touch screen to collect further input or responsive feedback from the athlete (e.g., operating as a sensor 102).

The respective rehabilitation device 104 may include, for example, an exercise machine, aerobic platform, or the like. In this regard, the athlete may operate the rehabilitation device responsive to receiving a prompt from the stimulus device 108 or a clinician. In some implementations, rehabilitation device 104 may include electronic circuitry and/or a processor that communicates exercise data (e.g., force and/or speed data) to computing device 106 during facilitation of a physical performance by the athlete. This data may be combined with sensor data from sensors 102 to provide a holistic assessment of the athlete's rehabilitation progress.

According to various implementations, sensor devices 102 include one or more EEG sensor(s), ECG sensor(s), EMG sensor(s), ACCL sensor(s), a respiration monitor, heart monitor, galvanic skin response device or electrodes, oxygen monitor, and/or a peripheral temperature sensor. Such sensors may embodied in, for example, one or more wearables such as a watch, chest or arm or thigh band, head unit, glasses, or combination thereof. Accordingly, the sensor devices 102 may collect physiological data including, for example, heart rate, heart rate variability, SpO2, respiration rate, ECG/EKG data, EMG data, galvanic skin response, or peripheral temperature. Similarly, the sensor devices may collect neurological data including, for example, spectral power bands, absolute or relative EEG power, neurological connectivity, EEG delta data, EEG theta data, EEG alpha data, EEG SMR data, EEG beta data, task load metrics, or attention metrics.

According to various implementations, EEG data may be collected using a EEG headset. In an example implementation, an EEG headset may include 21 channels of EEG and 2 channels of ExG. Data may be streamed to computing device 106 over a wired connection, or streamed wirelessly, for example, via Bluetooth. The headset may include built-in LED impedance measurements for each channel, and may include on-board impedance checking system for instant confirmation for quality of sensor connection.

EEG/qEEG may provide information concerning brain activation that will reflect the benefits of rehabilitation. The rationale for this hypothesis is that the benefits will be reflected through brain wiring systems such as activation of the Mirror Neurons. Further, Mirror Neuron Network (MNN) activation may provide an effective modality in post-surgery rehabilitation of lower extremities. For example, physical training of the contralateral limb may result in increased MNN activity in the lobe controlling the ipsilateral (injured) limb. Through neuro-rehabilitation training of the injured limb therapists may then precede and accelerate physical rehabilitation. Moreover, pre-injury measurement of MNN activity may provide a baseline from which to formulate training protocols and assess the progress of rehabilitation. By measuring MNN activity in athletes at various stages of lower extremity rehabilitation, the potential impact of MNN in rehabilitation may be assessed, facilitating a multifactor assessment for the safe return to play measures.

According to some implementations, the disclosed system may utilize preprocessed EEG. Preprocessed EEG includes raw signal data that has been filtered to remove artifact (such as ECG, EOG and EMG). Preprocessed EEG may support electrophysiological monitoring, and may be used to access brain activity, functional changes, neural networks and brain activation patterns.

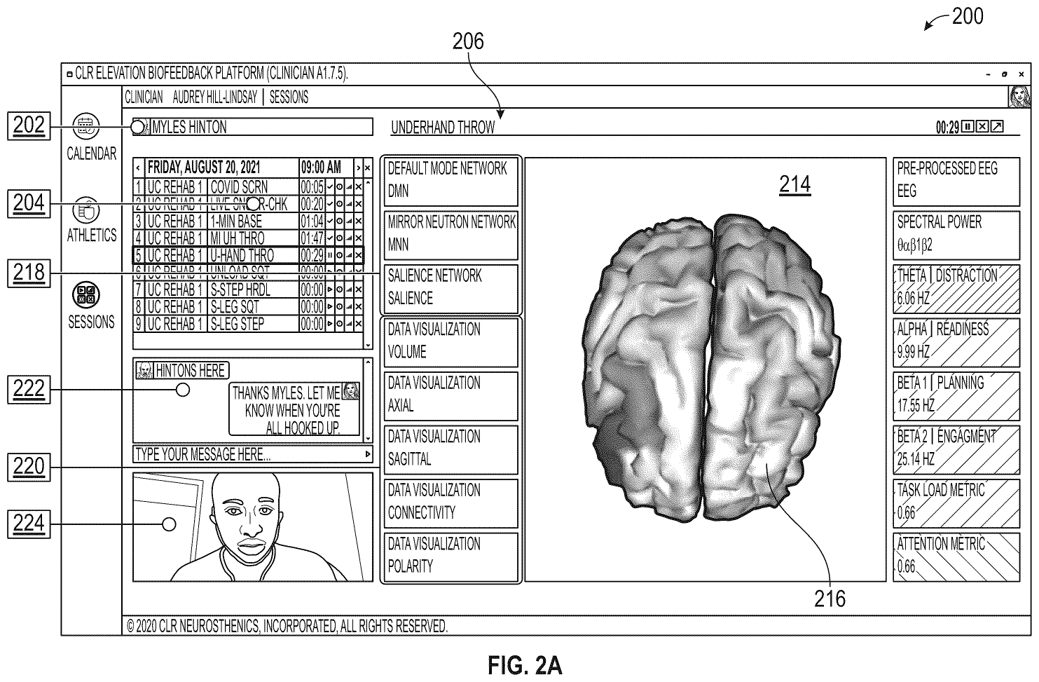

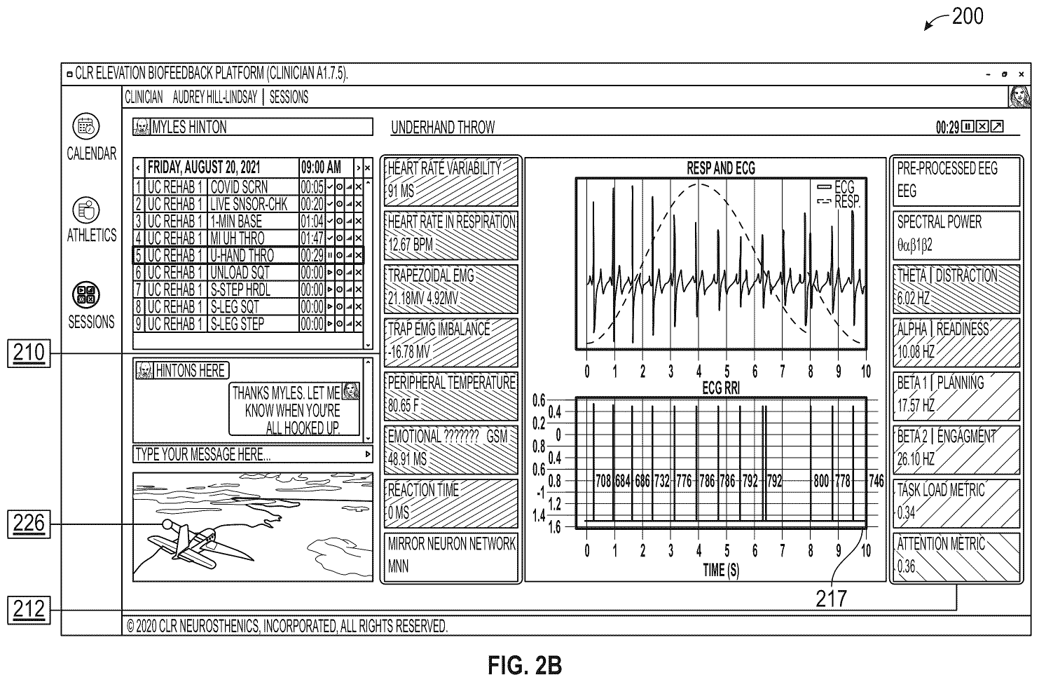

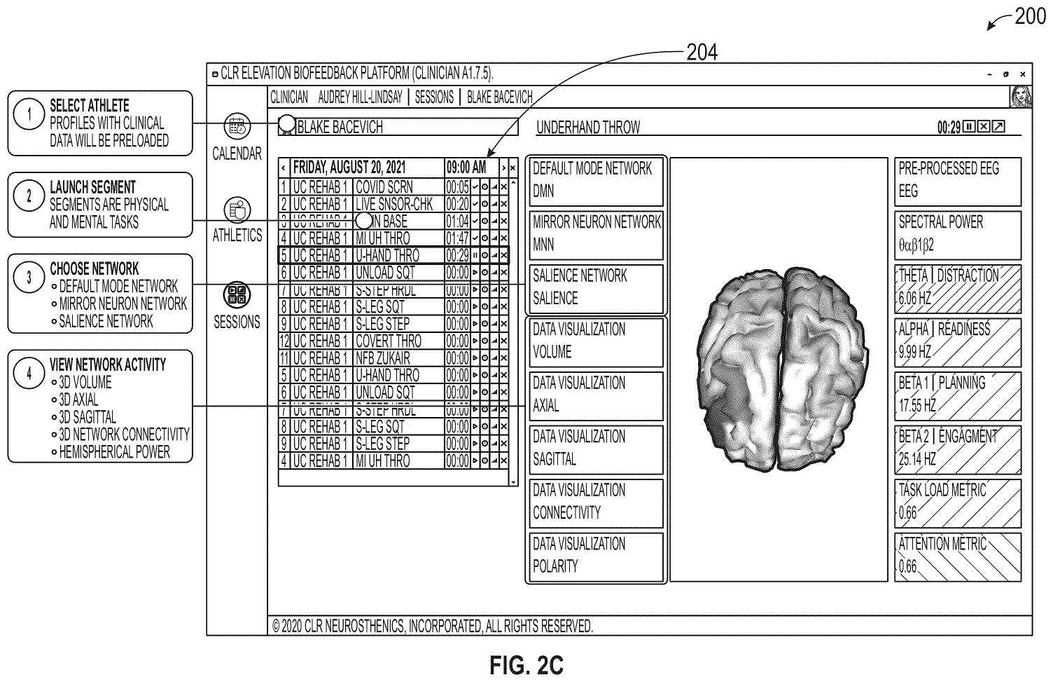

In some implementations, centralized server 112 may function as a web server, and the previously described virtual interface may be rendered from a website provided by server 112. According to various implementations, centralized server may further aggregate real time patient data and provide the data for display by computing device 106 (see, e.g., FIG. 2). The data may be provided (e.g., transmitted) for display by a clinician device, and the clinician device (including, e.g., computing device 106) may include a software client program or other instructions configured to, when executed by one or more processors of the device, render and display a graphical user interface (such as shown in FIG. 2) with the corresponding data.

FIGS. 2A, 2B, and 2C depict an example biometric interface 200 for initiating and controlling patient physiological and neurological rehabilitation, according to various aspects of the subject technology. Biometric interface 200 may be provided for display (e.g., on a computer monitor or other display device) by computing device 106 or server 112 (operating, e.g., as a web server). The subject biometric interface 200 provides a clinician multimodal, neuro-plastic assessment and training protocols that facilitate injury rehabilitation and prevention, and asses and maximize athletic performance. In this regard, as physiological and neurological data are collected from an athlete using the system of FIG. 1, the collected data is analyzed to identify the impact of the athlete's mental status (e.g., state of mind) on the athlete's physical capabilities and displayed on the subject interface. The interface 200 may then be used by a clinician to further control prompts provided to the athlete to initiate a physiological or neurological response, which is then assessed by sensors 102 and collected by the interface 200.

The biometric interface 200 may include an athlete selection screen whereby individualized data may be entered for a particular athlete, which may then be stored in a database (e.g., in database 110). Data collected by the system may include, for example, demographic data, medical history, as well as individualized profile data, including:
Age, Race, Ethnicity, Nationality, HHI+
Birthplace, Address, HUD ACS Scores
Education History, Current Enrollment
Weight, Measurements, Other Biometrics
Psychographics, Psychometrics
Sport, Position, Performance Statistics
NCAA ISP History (participation required)
Personal, Family Medical Histories
Known Conditions, Ailments
Past, Current Prescriptions
Behavioral Data
Clinical Notes+Next Protocol

The system may assign a unique identifier to the athlete so that the data may be stored as de-identified data. In some implementations, where de-identified data is not required (e.g., within a healthcare facility), the unique identifier may be used to aggregate de-identified data with identified data so that the interface may be personalized to the athlete. In other environments, for example, when evaluating the athlete's performance offsite, the interface may not have access to the identified data and the interface may only identify the athlete by the unique identifier or other HIPAA compliant designation.

Other information collected by the system to define a schedule of tailored assessment and training sessions include:
Athlete ID
Trainer
Clinician
Date, Time
Location
Injury Type
Session Type
Planned Segments
Segment Goals
Summary Results
Clinical Notes

As depicted in the example of FIGS. 2A and 2B, once an athlete is selected for evaluation and rehabilitation, the clinician interface 200 may include an identification section 202, a task section 204, and a data section 206. Data section 206 may display different data modalities, depending on which task is selected and/or other selections made by the clinician. FIG. 2A depicts display of brain function data. In this regards, interface 200 may include a physiological data section 210, a neurological data view section 212, and a graphical data view section 214. The graphical data view section 214 may display a brain map 216 (FIG. 2A) when neurological data is being analyzed and physiological data 217 (e.g., waveforms) when physiological data is being analyzed. Interface 200 may include a selection control (not shown) for selecting an injury type (e.g., left ACL injury) and for switching between data modalities for treatment of the injury type. When neurological data is selected, interface 200 may further include a set of network controls 218 and a set of brain view controls 220. Selection of controls 218, 220 may change what portions of the brain map 216 are displayed. As shown in FIG. 2A, certain areas of the brain map may be highlighted according to neurological metrics (e.g., power levels) measured within the brain (e.g., using sensor(s) 102). Additionally or in the alternative, areas may be highlighted based on data for those areas (e.g., regions and/or lobes of the brain) meeting certain criteria, such as having a measured value greater than a particular threshold or falling within certain data ranges. In some implementations, as shown in FIG. 2B, physiological data section 210 may be displayed when physiological data is selected for display.

During each athletic assessment and/or training session, the system may further place event markers to indicate which physical or mental exercise was performed during the recording of the following neuro-physiological data:
Processed qEEG Channels
qEEG Band Power & Hz
Spectral Power, Connectivity and Asymmetry of the Mirror Neuron Network,
Salience Network and Default Mode Network
SMR, Coherence, Phase & Amplitude
Attention & Task Load Metrics
ERP & P300
Stress & Recovery
Heart Rate & Heart Rate Variability
Respiration Rate & SpO2
Trapezoidal, Frontalis and other EMG
Galvanic Skin Response and Peripheral Temperature

During each athletic assessment and/or training session, the system may also record applicable, session-related data, including:
Pre-Session Vital Statistics
Pre-Session Survey Responses
Pre-session Risk Propensity & Task Confidence Levels
Pre-session Neurovisual Tracking Ability
In-Session Reaction Times
In-Session Stroop Time & Accuracy
In-Session Neuro-Feedback Game/Test Scores
In-Session Biometrics (collected from external devices)
In-Session Clinical Notes
Post-Session Composite Analytics

Interface 200 may further include a live chat area 222 and webcam area 224 so that a clinician may interact with the patient remotely. In some implementations, interface 200 may further include an patient monitor section 226 which may include a representation of a stimulative visualization currently being provided to the patient (e.g., via stimulus device 108). The stimulative visualization may, for example, include a graphic that adjusts to a target based on patient neurological and/or physiological activity, thereby providing a real-time indication of a deviation from a performance target. In the depicted example, an airplane is shown. As the patient changes focus on the performance target, the stimulative subject (e.g., the airplane) may adjust towards or away from the target depending on the patient's focus and/or attention. When the collected neurological or physiological data is within a target range, the airplane's course may adjust to maintain a steady course. However, the airplane may deviate from the course an amount corresponding to an amount of the data's deviation from the target range. In another example, a pacer visualization may move up and down to stimulate the patient to match their respiration rate to that of the pacer.

FIG. 2C depicts an example process for initiating and performing patient physiological and neurological using the biometric interface. According to various implementations, a clinician may log on to the user interface 200 and (1) select a patient from a menu within identification section 202 (e.g., FIG. 2A), at which point the software may query the database 110 for stored profile and session data associated with the selected patient. Task section 204 may include a list of available task segments for the patient. Each task segment may include one or more physical or mental tasks to be performed by the athlete. According to various implementations, a task or group of tasks, or rehabilitation protocol (see, e.g., table A, B, C, D), may be selected by the system based on the patient's injury type.

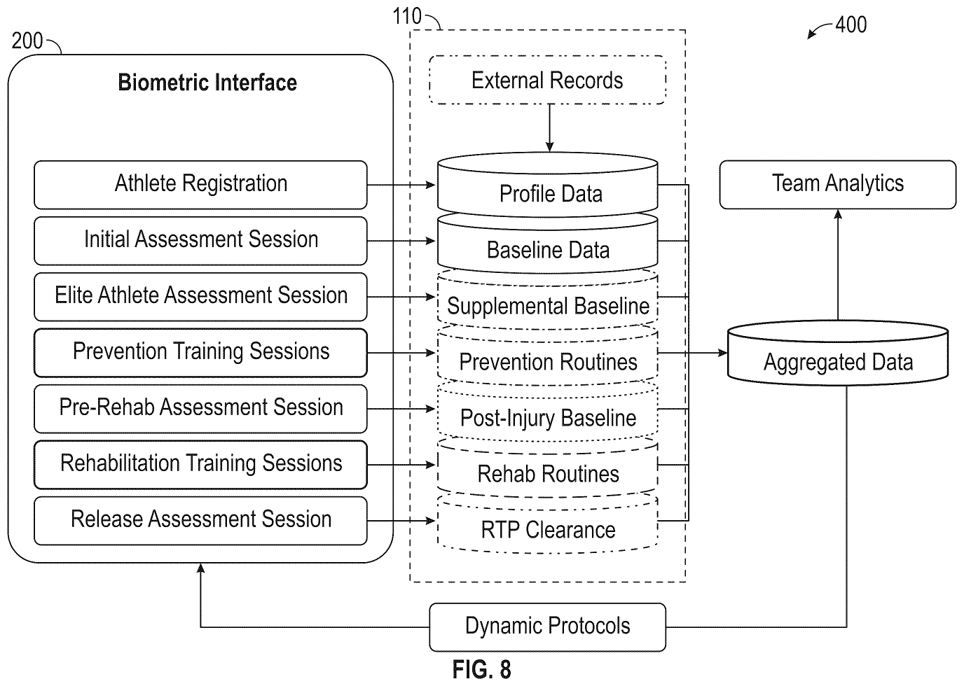

The clinician may then (2) launch a segment by selecting a segment from task section 204. Selection of a task segment may cause the system to reconfigure the interface 200 to display one or more neurological and physiological configuration selections, allowing the clinician to visually target certain areas of the brain through neurological signals received from the EEG headset or from physiological signals received from various sensors attached to the body. In some implementations, the clinician navigates to the neurological network controls 218 and (3) chooses one or more neurological networks to target with the biometric interface. In some implementations, the system will automatically select a network based on the type of injury. The clinician may then (4) select various data views for display in graphical data view section 214, for example, in brain map 216 to view results of data assessed by the system. FIGS. 8A through 8G depict example neurological data assessments and visualizations that may be produced by the biometric interface 200. FIGS. 9A through 9C depict example physiological data assessments and visualizations that may be produced by the biometric interface 200.

As depicted in FIGS. 2A and 2B, the biometric interface 200 may display a list of tasks selected for the selected patient. Each task segment may include one or more physical and/or mental tasks which the patient performs while being monitored by the sensors 102 to establish a baseline neurological assessment or to determine progress of rehabilitation. These physical and/or mental tasks may be performed by the patient during one or more predetermined overt or covert assessment exercises. A rehabilitation protocol that includes baseline assessment segments may include the following rehabilitation segment tasks:

TABLE A

| # | SEGMENT TASK | TIME | DESCRIPTION |
|---|---|---|---|
| 1 | COVID Screen | 10 | |
| 2 | Live Sensor Check | 60 | |
| 3 | qEEG- Eyes Closed (EC) | 60 | |
| | qEEG- Eyes Open (EO) | 60 | |
| | qEEG- Visual Arrest | 120 | |
| 4 | Stroop | 120 | 2 sets -- Right/Left |
| 5 | Recovery (EO) | 60 | Recovery from stressor: ideal to |
| | | | get back to 75% of baseline |
| 6 | Positive Imagery | 45 | Positive stressor: the ability |
| | | | to use imagery for learning and |
| | | | visualization |
| 7 | Recovery (EO) | 60 | Recovery from stressor: ideal to |
| | | | get back to 75% of baseline |
| 8 | Anticipation | 42 | Measures stress response & |
| | | | arousal level to anticipation |
| | Brief Stressor | 3 | Measures stress response & |
| | | | arousal level to stressful |
| | | | stimuli |
| 9 | Recovery (EO) | 60 | Recovery from stressor: ideal to |
| | | | get back to 75% of baseline |
| 10 | Motor Image (MI) | 107 | Pre Motor Task |
| | Underhand Throw | | |
| 11 | Underhand Throw | 107 | |
| 12 | Unloaded Squat | 20 | 3 sets up and down slowly |
| 13 | Side-Step Hurdle (Right) | 15 | 3 sets |
| 14 | Side-Step Hurdle (Left) | 15 | 3 sets |
| 15 | Single-Leg Squat (Right) | 10 | 3 sets |
| 16 | Single-Leg Squat (Left) | 10 | 3 sets |
| 17 | Single Leg Step-Down | 20 | 3 sets |
| | (Right) | | |
| 18 | Single Leg Step-Down | 20 | 3 sets |
| | (Left) | | |
| 19 | Reaction Time | 300 | 2 sets (R/L): Measuring response |
| | | | time based upon stimuli |
| 20 | Recovery (EO) | 60 | Recovery from stressor: ideal to |
| | | | get back to 75% of baseline |
| 21 | Underhand Throw | 107 | |
| 22 | Unloaded Squat | 20 | 3 sets up and down slowly |
| 23 | Side-Step Hurdle (Right) | 15 | 3 sets |
| 24 | Side-Step Hurdle (Left) | 15 | 3 sets |
| 25 | Single Leg Squat (Right) | 10 | 3 sets |
| 26 | Single Leg Squat (Left) | 10 | 3 sets |
| 27 | Single Leg Step Down | 20 | 3 sets |
| | (Right) | | |
| 28 | Single Leg Step Down | 20 | 3 sets |
| | (Left) | | |
| 29 | MI Underhand Throw | 107 | Post Motor Task |
| | Total Time | 508 | Seconds |
| | | 28.47 | Minutes |

An alternative baseline assessment task list may include the following segment tasks:

TABLE B

| # | SEGMENT TASK | TIME |
|---|---|---|
| 1 | qEEG- (Eyes Closed) | 00:00:60 |
| 2 | qEEG- (Eyes Open) | 00:00:60 |
| 3 | qEEG- Visual Arrest | 00:02:00 |
| | Stroop Test | 00:02:00 |
| | Recovery (Eyes Open) | 00:00:60 |
| 4 | Positive Imagery | 00:00:45 |
| 5 | Recovery (Eyes Open) | 00:00:60 |
| 6 | Anticipation | 00:00:42 |
| 7 | Brief Stressor | 00:00:03 |
| 8 | Recovery (Eyes Open) | 00:00:60 |
| | MI Underhand Throw | 00:01:47 |
| 9 | Underhand Throw | 00:01:47 |
| 10 | Unloaded Squat | 00:00:20 |
| 11 | Side-Step Hurdle (Right) | 00:00:15 |
| 12 | Side-Step Hurdle (Left) | 00:00:15 |
| 13 | Single Leg Squat (Right) | 00:00:10 |
| 14 | Single Leg Squat (Left) | 00:00:10 |
| 15 | Single Leg Step-Down (Right) | 00:00:20 |
| 16 | Single Leg Step-Down (Left) | 00:00:20 |
| 17 | Reaction Time | 00:05:00 |
| 18 | Recovery (Eyes Open) | 00:01:00 |
| 19 | Underhand Throw | 00:01:47 |
| 20 | Unloaded Squat | 00:00:20 |
| 21 | Side-Step Hurdle (Right) | 00:00:15 |
| 22 | Side-Step Hurdle (Left) | 00:00:15 |
| 23 | Single Leg Squat (Right) | 00:00:10 |
| 24 | Single Leg Squat (Left) | 00:00:10 |
| 25 | Single Leg Step-Down (Right) | 00:00:20 |
| 26 | Single Leg Step-Down (Left) | 00:00:20 |
| 27 | MI Underhand Throw | 00:01:47 |

Supplemental assessment segments may include:

TABLE C

| # | SEGMENT TASK | TIME | DESCRIPTION |
|---|---|---|---|
| | 1-min Baseline | 60 | |
| | Knee Laxity | NA | Open ended recording with Event |
| | | | Markers |
| | Lower Leg Range | NA | Open ended recording with Event |
| | of Motion | | Markers and ROM Degrees |
| | Lower Leg | NA | Open ended recording with Event |
| | Resistance | | Markers and force NMs |
| | Gait Exercises | NA | Open ended recording with Event |
| | | | Markers and performance indices |
| | Force Plate | NA | Open ended recording with Event |
| | Exercises | | Markers and force NMs |
| | Balance Exercises | NA | Open ended recording with Event |
| | | | Markers and performance indices |
| | 2D & 3D Light | NA | Open ended recording with Event |
| | Stimulation | | Markers and physical response |
| | | | indices |
| | Weight Exercises | NA | Open ended recording with Event |
| | | | Markers and performance indices |
| | Motor Task R | NA | Open ended recording with Event |
| | | | Markers |
| | Motor Task L | NA | Open ended recording with Event |
| | | | Markers |

On selection and/or initiation of a respective task, the software selects and connects the corresponding sensors 102 associated with the selected tasks, causes or facilitate causing a prompting of the tasks to be performed by the athlete (e.g., via stimulus device 108) in the specified order, causes the collection of data from the sensors while the tasks are being performed, and analyzes and stores the data obtained from the sensors 102. Additionally or in the alternative, a respective task may be selected individually from the task list.

During neurologic data collection, each subject may participate in one or more of the following covert and overt exercises:

TABLE D

| | EXERCISE | DESCRIPTION |
|---|---|---|
| 1 | Eyes Closed | 1 minute EEG recording using CGX Quick-20r cap |
| | 1-Minute | with eyes closed |
| | Baseline | |
| 2 | Stroop Test | Responsive task of indicating the actual color used to |
| | | display a particular word on the screen. For example, |
| | | the word “blue” might be displayed in red, but the |
| | | individual is asked to say the color of the word, in |
| | | this case blue, rather than the word itself - in this |
| | | case red. |
| 3 | Covert | Visualization of throwing a ball underhanded, |
| | Under- | alternating between R and L hands with eyes closed. |
| | Handed | A sound will play to indicate when to visualize a |
| | Throw (EC) | throw. There will be 12 visualizations per minute, 36 |
| | | in total. |
| 4 | Overt | Motor task of throwing a ball underhanded, |
| | Under- | alternating between R and L hand, while standing |
| | Handed | with eyes open. Will see a start in center of screen to |
| | Throw | focus on, then when screen changes to Red, throw |
| | | with your R hand, and when it changes to Green, |
| | | throw with your L hand. There will be 12 |
| | | visualizations per minute, 36 in total. |
| 5 | Motor Task - | Motor task of an unloaded squat with arms overhead, |
| | Unloaded | bending knees 90 degrees, hold for ½ second, |
| | Squat | straighten and rest for 30 seconds. Repeat 2 more |
| | | times, 3 sets in total. |
| 6 | Motor Task - | Motor task of a side-step hurdle. Stand to side of |
| | Side-Step | hurdle with both legs to R of hurdle, step over hurdle |
| | Hurdle | with R leg, leaving room for L foot, lift L leg over |
| | | hurdle next to R leg, pause, put back over and repeat |
| | | 2 more times, 3 sets in total. Repeat with L leg to L |
| | | side of the hurdle, bringing the R leg over next to it |
| | | for a total of 3 sets. |
| 8 | Motor Task - | Motor task of single leg step down. Standing on a |
| | Single Leg | box with weight evenly distributed on both legs and |
| | Step Down | nothing in hands, pick L leg up off the box and |
| | | slowly flex R leg, put L leg on ground, then bring R |
| | | leg down on ground next to L leg, redistribute weight |
| | | evenly between legs, then bring both feet back onto |
| | | box, repeat 2 more times for a total of 3 sets. Do the |
| | | same thing by picking up R leg and slowly flexing L |
| | | leg for a total of 3 sets. |
| 9 | Covert | Visualization of throwing a ball underhanded, |
| | Under- | alternating between R and L hand with eyes open. |
| | Handed | Will see a start in center of screen to focus on, then |
| | Throw EEG | when screen changes to Red, throw with your R |
| | NFB (EO) | hand, and when it changes to Green, throw with your |
| | | L hand. There will be 12 visualizations per minute, |
| | | 36 in total. |
| 10 | EEG | Will be flying a plane by using brainwaves. |
| | Neuro- | |
| | feedback | |
| 11 | Overt | Motor task of throwing a ball underhanded, |
| | Under- | alternating between R and L hand, while standing |
| | Handed | with eyes open. Will see a start in center of screen to |
| | Throw | focus on, then when screen changes to Red, throw |
| | | with your R hand, and when it changes to Green, |
| | | throw with your L hand. There will be 12 |
| | | visualizations per minute, 36 in total. |
| 12 | Motor Task - | Motor task of an unloaded squat with arms overhead, |
| | Unloaded | bending knees 90 degrees, hold for ½ second, |
| | Squat | straighten and rest for 30 seconds. Repeat 2 more |
| | | times, 3 sets in total. |
| 13 | Motor Task - | Motor task of a side-step hurdle. Stand to side of |
| | Side-Step | hurdle with both legs to R of hurdle, step over hurdle |
| | Hurdle | with R leg, leaving room for L foot, lift L leg over |
| | | hurdle next to R leg, pause, put back over and repeat |
| | | 2 more times, 3 sets in total. Repeat with L leg to L |
| | | side of the hurdle, bringing the R leg over next to it |
| | | for a total of 3 sets. |
| 14 | Motor Task - | Motor task of a single leg squat. With arms overhead, |
| | Single Leg | transfer all weight to R leg by bending L flexed |
| | Squat | under body so center of gravity is unchanged, hold 1 |
| | | second once not wobbling, then squat by flexing |
| | | knee to 90 degrees, hold 1 second, then straighten leg |
| | | until upright again and hold until no more wobble, |
| | | repeat 2 more times, 3 sets in total. Do the same |
| | | thing with weight transferred to the L leg for a total |
| | | of 3 sets. |
| 15 | Motor Task - | Motor task of single leg step down. Standing on a |
| | Single Leg | box with weight evenly distributed on both legs and |
| | Step Down | nothing in hands, pick L leg up off the box and |
| | | slowly flex R leg, put L leg on ground, then bring R |
| | | leg down on ground next to L leg, redistribute weight |
| | | evenly between legs, then bring both feet back onto |
| | | box, repeat 2 more times for a total of 3 sets. Do the |
| | | same thing by picking up R leg and slowly flexing L |
| | | leg for a total of 3 sets. |
| 16 | Covert | Visualization of throwing a ball underhanded, |
| | Under- | alternating between R and L hands with eyes closed. |
| | Handed | A sound will play to indicate when to visualize a |
| | Throw | throw. There are 12 visualizations per minute, 36 in |
| | | total. |

The subject technology generates physical and/or mental tasks than not only facilitate recovery of a physical injury but reinforce recovery by way of neurological pathways and neuroplasticity. In this regard, the system identifies neurological changes in areas of the patient's brain associated with the injury and targets those areas for rehabilitation through mental and physical tasks and/or exercise (in coordination with physical rehabilitation). A pre-injury state of the patient is modeled, either through training data based on a population, or by way of pre-assessment before injury, and a baseline is developed.

Accordingly, as a patient goes through a rehabilitation protocol the system may observe and adapt to changes in neuroplasticity. For example, when an athlete is injured, the regions within the patient's brain may become asymmetrical. As the athlete is rehabilitated according to aspects of the subject technology, brain symmetry improves, which greatly affects the patient's athletic ability.

The system identifies tasks and/or stimulus and/or a rehabilitation protocol that improves the target neuroplasticity, and continues to apply it. In this regard, the system obtains a brain map 216 using sensors 102. According to various implementations, the brain map includes or is provided in connection with neurological data and data bands such as EEG, qEEG, ExG, and ECG data. Certain EEG bands may be indicative of certain deficiencies and/or proficiencies in measured neurological regions of the brain.

According to various implementations, multiple predetermined regions (e.g., six) may be identified in each hemisphere in the brain. Utilizing the map 216, the system may measure one or more neurological metrics, including brain connectivity, activation, and/or symmetry, among other things, within each of one or more target regions for one or more networks within the brain, including Mirror Neuron Network (MNN), Salience Network, and/or the brain's Default Network. The system then determines a change of each of the target neurological metrics from the baseline (e.g., pre-injury) and generates activities for the patient that drive the neurological metrics (and thus, target neuroplasticity) back towards the baseline.

The Mirror Neuron Network links sensory stimuli to the motor system for control of physical actions. These neurons mirror the actions and behaviors of their contralateral counterparts, providing the neural basis for understanding contralateral movement, and encoding the meaning of contralateral actions, for example when one arm signals the other arm while performing an activity. When a person throws a ball from the left arm to the other arm without looking the MNN is responsible for communicating the speed, force, movement to areas of the brain that control the other arm so that those regions know how to adjust the other arm to catch the ball.

The Salience Network of the brain selects which stimuli to focus and pay attention to. It includes a collection of regions of the brain that pick up stimuli and inform the person whether the person should pay attention to it or not, supporting complex functions such as communication and self-awareness. The network has key nodes in the insular cortex and is critical for detecting behaviorally relevant stimuli and for coordinating the brain's neural resources in response to these stimuli. The Salience Network also integrates sensor, emotional, and cognitive information, and is generally more trainable than other networks.

The Default Network includes a collection of interconnected structures and regions of the brain that analyze and process information while the brain is idling; that is, when a person is awake, but not engaged in any particular mental exercise. These regions are more active during passive tasks than tasks demanding focused external attention. The Default Network is used all the time—not only for rest, but also for recall and planning. Training the Default Network makes other networks more receptive to training. In this regard, the system may target the Default Network as one in series of targeted network to reinforce training, or to prepare for training, of regions in the other networks.

As will be described further, the foregoing networks may be trained by way of protocols that involve a form of neuro-regulation/neuro-physical training. In this regard, the system targets neurological metrics such as connectivity, activation, and/or symmetry within each network. Connectivity is the speed and efficiency with which the lobes/regions (neurons) of the brain are communicating with each other. Connectivity indicates the firing rates and response time of neurons in the cortex within a specified network (e.g., MNN, Salience, Default, etc). Firing rates are modulated by changes in brain states, such as various states of attention or information processing.

The system analyzes nodes that are firing in the various regions and, in comparing to baseline, determines whether nodes are firing properly, experiencing a level of difficulty, or not firing. Connecting may be measured by coherence, phase and amplitude. In this regard, the system may determine regions of the brain that are overcompensating for dysregulated regions. In some implementations, connectivity may provide a level of inhibition in the patient. The level of connectivity in a certain area of the brain may be delineated, e.g., in the user interface, by different colors of the node and/or the connections.

According to various aspects, if an athlete is injured then the contralateral side will likely exhibit less connectivity. The system detects this deficiency by comparing the detected connectivity with a baseline connectivity for the region and generates activities that facilitate increasing connectivity in the affected region.

From the brain map 216 and analytics, the system can determine what regions of the brain have poor connectivity. The region may be too fast (e.g., hyper) or too slow (e.g., hypo), which may be displayed as red or blue. A protocol is selected to change the connectivity more seamless and/or improve it in one or more of the identified regions.

If connectivity is slower in a region; e.g., from a concussion or TBI, a power issue may be identified and a rehabilitation protocol may be generated to speed up those connections over time to improve symmetry. In this regard, the data returned by the system (e.g., in a brain map) may show what can be changed and or how much can be changed (based on a baseline), and generate the appropriate therapy to improve the connectivity in the targeted region(s).

The system may also measure activation, or power output, in selected regions of the brain associated with the injury. Accordingly, the system may measure spectral power, which may be indicative of a shift from lower to higher frequency ranges measured as relative power between the right and left hemispheres. A difference in power output may also be observed between the corresponding regions in the left and right hemisphere. Relative power may also be indicated by regions and/or lobes within each hemisphere. Thus, overcompensation in a particular region that results from an injury may be determined based on comparing current measurements with the baseline. They system may then generate tasks and/or activities for the patient that facilitate improvement in activation for the selected regions.

They system also employs brain mapping to detect and visualize patterns of asymmetry between target regions of the respective left and right hemispheres of the brain. In this regard, the system may identify the balance or the imbalance or the regulation or dysregulation of brain activity in a contralateral side of the brain as an injury. For example, if an athlete injures his/her left knee then there should be asymmetry in the right cortex (opposite). The system may detect a level of asymmetry between the hemispheres for the region associated with the injury and provide a protocol for improving the symmetry in the affected area.

In one example, the system may initiate motor activity/exercises for each of multiple (e.g., six) regions prior to a patient's injury to establish a baseline for the patient with regard to select neurological metrics in the target regions. With regard to symmetry, the system may select regions with a focus on the MNN. For example, the system may particularly measure the MNN in the Sensorimotor rhythm (SMR) region (e.g., between 13-15 Hz). In some implementations, they system determines a level of symmetry between respective regions in each hemisphere based on the power output of the regions.

The object is to move the targeted regions of the patient's brain toward symmetry. Injuries and failures are known to exist when asymmetry is present. For example, a patient who is injured on his or her left side may exhibit signals that indicate the right side of the brain took over most of the function of the left side. As the patient is moved toward symmetry, the patient is expected to move towards recovery of the injury.

Regions are identified and selected according to the injury being evaluated. If a patient baseline for the patient is available—that is, the patient was measured with regard to these metrics pre-injury—then the patient baseline is used by the system to determine/select therapies to apply to the patient to drive the targeted neuro-metrics towards the baseline. If a patient baseline is not available, then the system may select a baseline for the neuro-metric based on patient training data for a predetermined patient population that correlates with the patient (e.g., similar characteristics, demographics, etc.).

FIG. 3A depicts a first example process 300 for physiological and neurological rehabilitation of an athlete, according to aspects of the subject technology. For explanatory purposes, the various blocks of example process 300 are described herein with reference to FIGS. 1, 2A, and 2B, and 4-9 and the components and/or processes described herein. The one or more of the blocks of process 300 may be implemented, for example, by a computing device, including a processor and other components utilized by the device. In some implementations, one or more of the blocks may be implemented apart from other blocks, and by one or more different processors or devices. Further for explanatory purposes, the blocks of example process 300 are described as occurring in serial, or linearly. However, multiple blocks of example process 300 may occur in parallel. In addition, the blocks of example process 300 need not be performed in the order shown and/or one or more of the blocks of example process 300 need not be performed.

In the depicted example, the system obtains first physiological and/or neurological data pertaining to athletic performance of a person reacting to predetermined visual stimulation (302). As will be described further with respect to FIG. 3B, the data may include measurements obtained by sensor(s) 102 pertaining to a neurological metric such as relative spectral power (e.g., activation), network connectivity, or symmetry of one or more regions of the athlete's brain. The regions may, for example, be selected based on the type of injury being rehabilitated. Additionally or in the alternative, audible stimulation may be provided. In some implementations, the data collection forms the basis for an initial assessment of the athlete to create a baseline from which the athlete (or athletes) can be further evaluated and trained to prevent further injury and/or to recover from injury before return to play. For example, if the athlete is assessed pre-injury then a baseline may be developed for the pre-injury state of the athlete with regard to the physiological and/or neurological areas to be evaluated and/or rehabilitated. In other examples, the baseline may be a current condition of the athlete post-injury, and the system may generate rehabilitation actions to move the athlete toward a baseline based on a predetermined non-injured population.

According to various implementations, the system generates task segments associated with the one or more actions, with each task segment including one or more physical or mental tasks to facilitate rehabilitation. The athlete is then prompted, either audibly (e.g., verbally) or visually, to performed the tasks. In one example, a prompt may include performing an activity when the screen changes to a particular color.

While the following examples are concerned with collecting of data through the biometric interface 200, and sensors 102, at least the first physiological and neurological data may be additionally or alternatively based on predetermined data provided to the system and/or training data collected from a population over a period of time.

During the rehabilitation of the athlete, the audible or visual stimulation provided to the athlete may be based on the task segment and include visual instruction for prompting the person to perform the one or more physical or mental tasks. As described previously, the data may be collected by initiating a baseline assessment protocol, including a list of segment tasks (as in, e.g., Table A or Table B). According to various implementations, the physiological and neurological data is obtained in real-time responsive to a performance of the physical or mental tasks by the athlete.

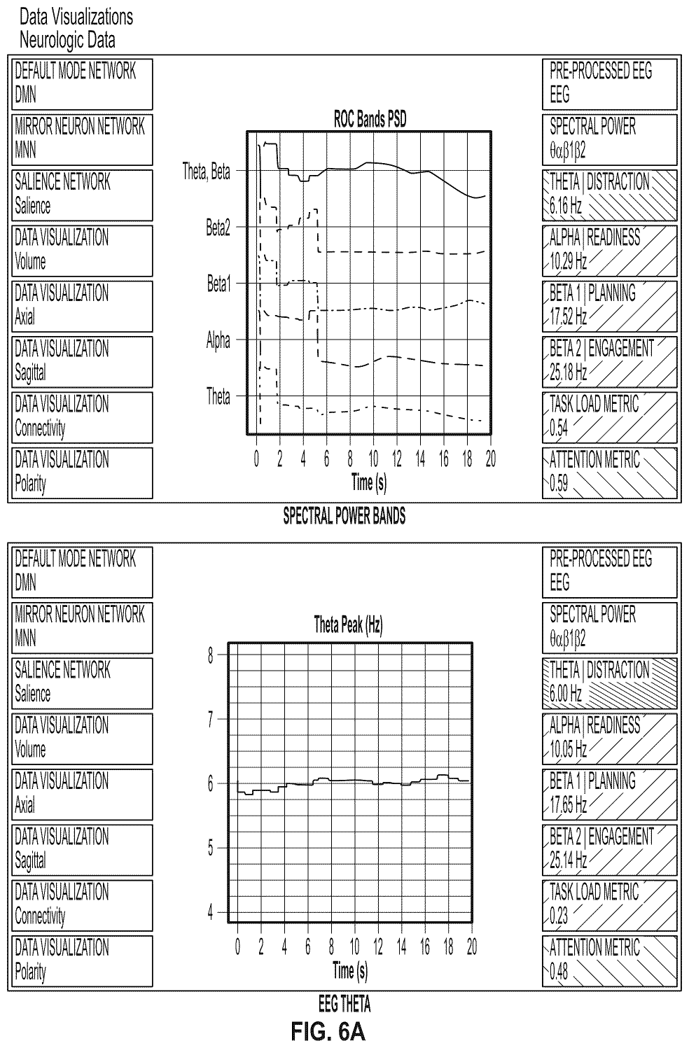

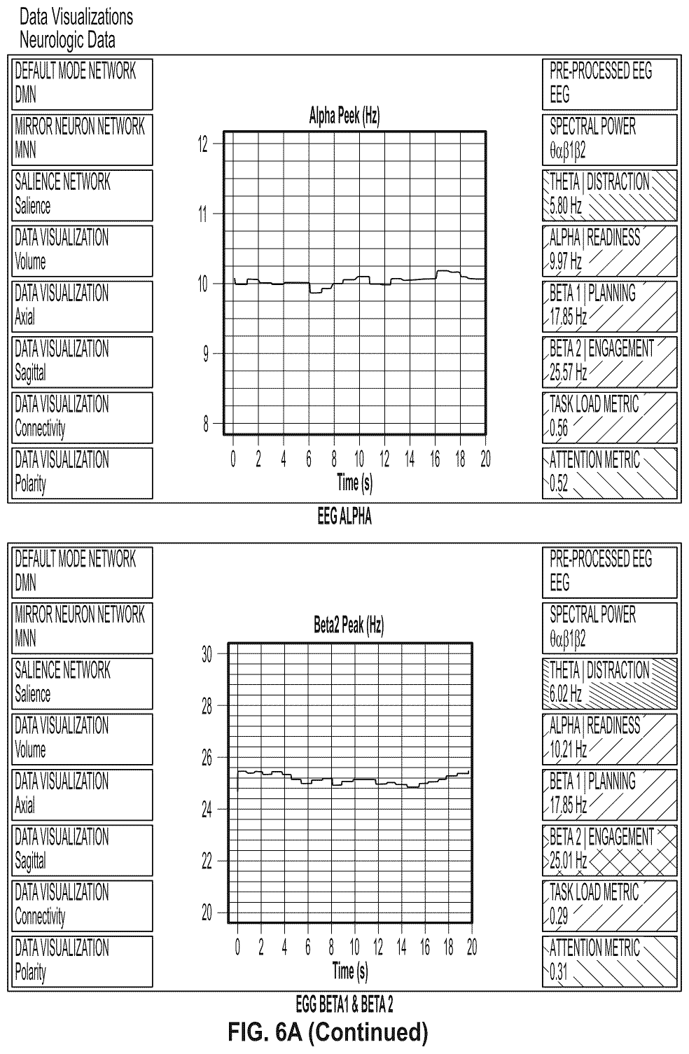

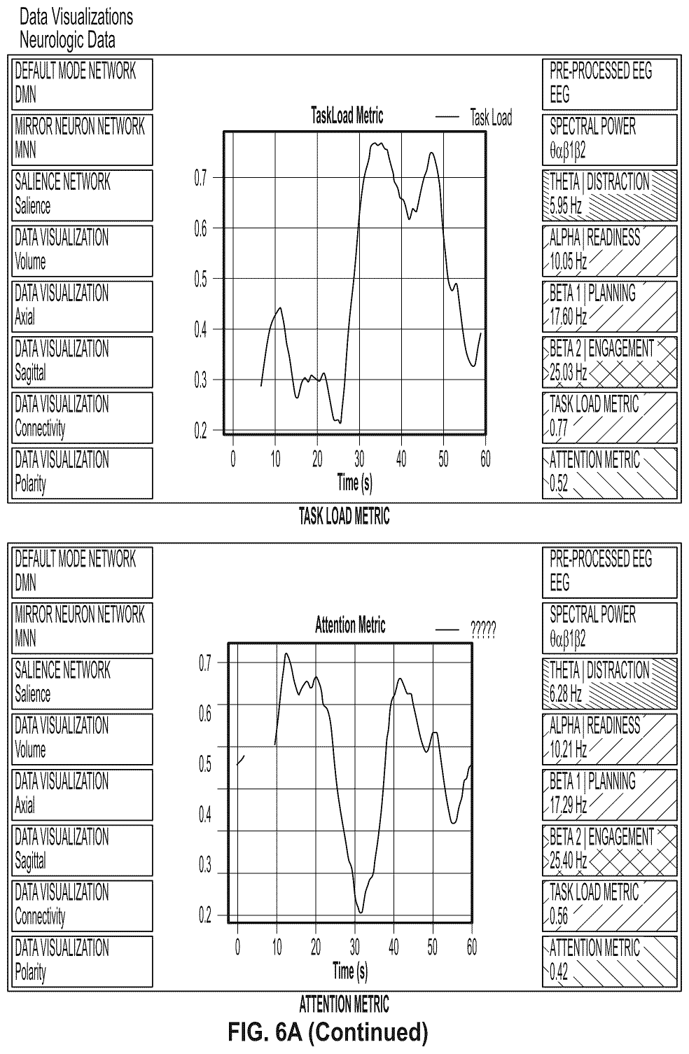

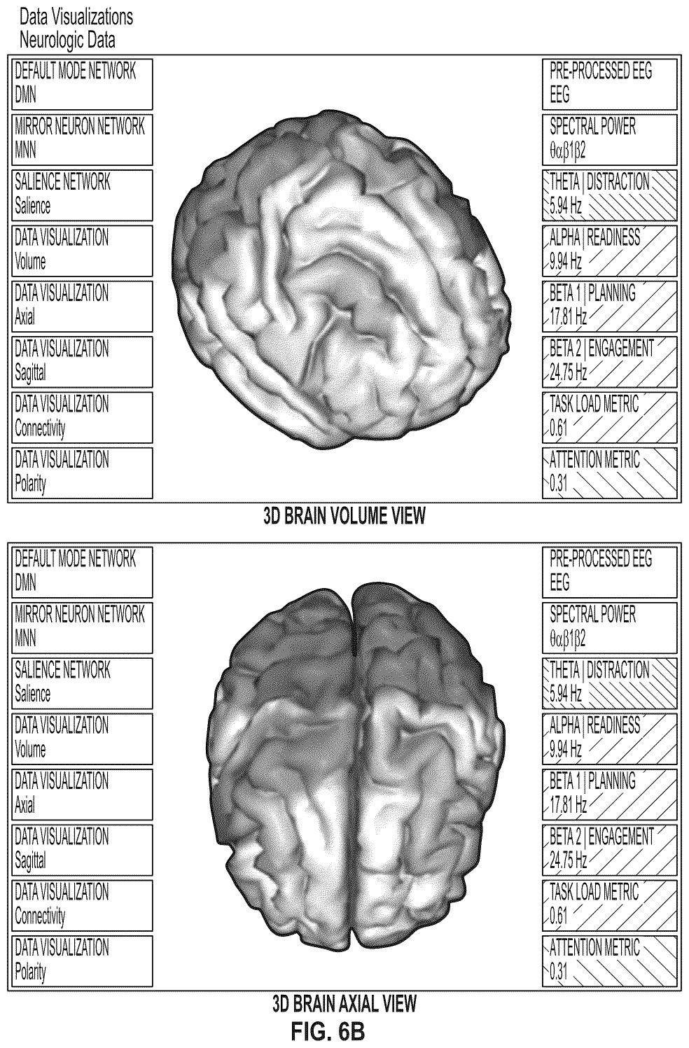

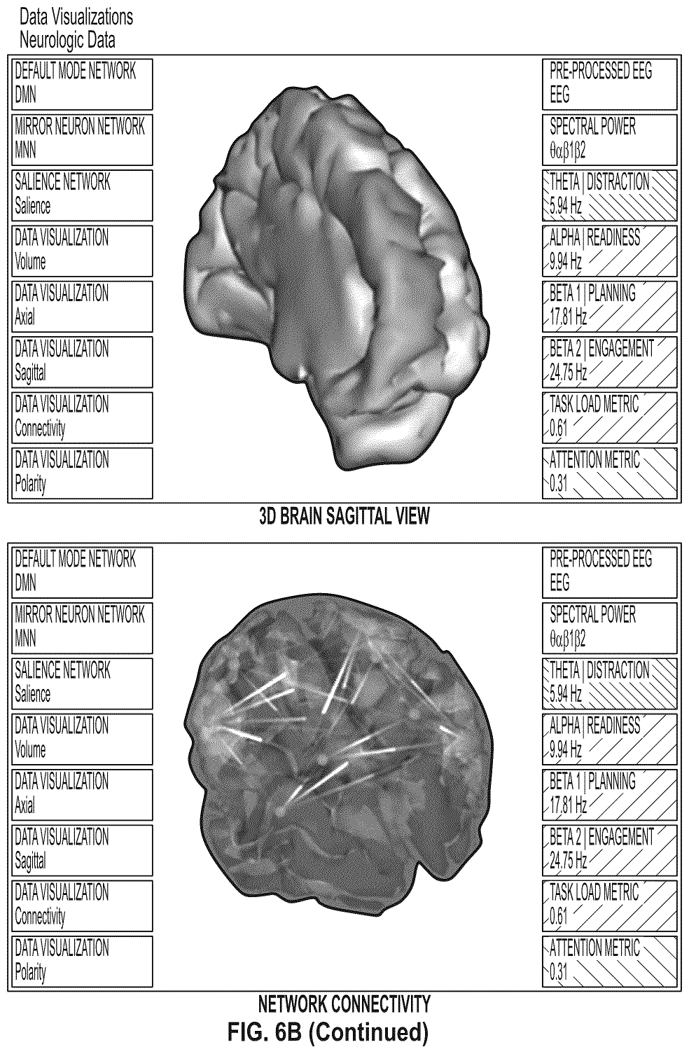

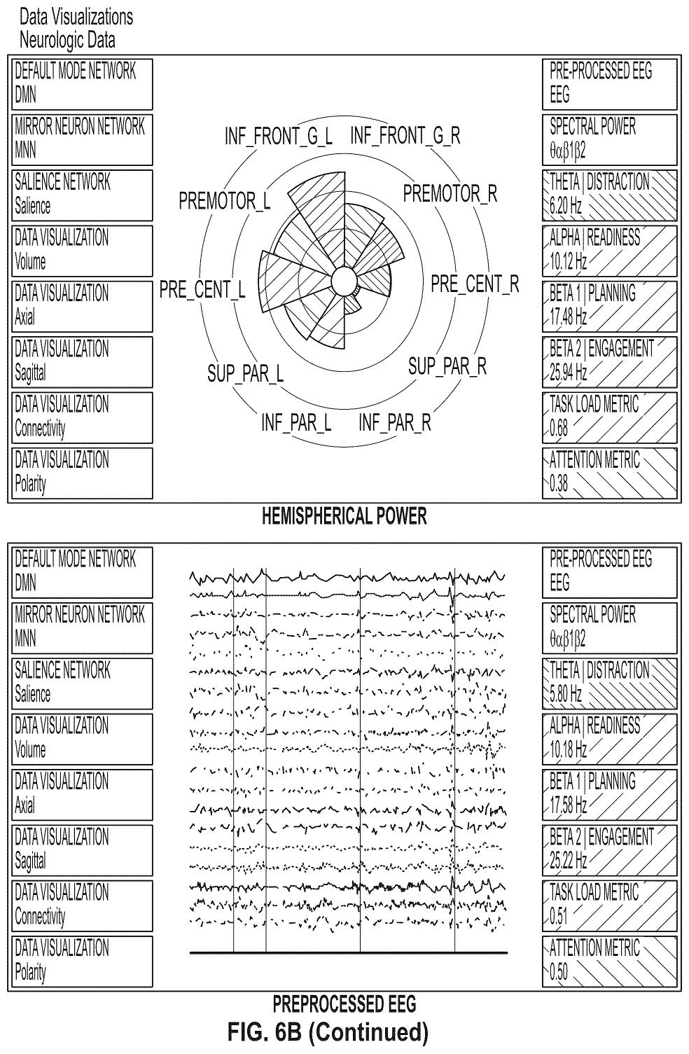

The assessment tasks provided by the system may be based on the established methods of the functional movement scale. In one example, the tasks may include overhead squat, single leg squat, step down, knee lax, and/or lateral hurdles. Exercises include those designed to stimulate specific neuro-physical response, and may be particularly useful during a baseline assessment. Exercises include motor tasks (e.g., stepping and/or aerobic exercise), motor imagery, iso-kinetic exercises, cognitive tests, and biofeedback, as indicated in FIG. 6.

An example assessment session may take, for example, about 36 minutes to complete, and may include placement of a qEEG headset (or cap). Visualization of activities are rendered by the biometric interface 200 responsive to stimulation (e.g., verbal instructions. Continuous data collection is achievable via the qEEG headset and other sensors 102, described previously.

According to various implementations, the physiological and neurological data is obtained by the system (via sensors 102 and/or devices 104) in real-time responsive to a performance of at least one physical or mental tasks by the athlete. The physiological data obtained by the system may include, for example, one or more of heart rate, hear rate variability, respiration rate, ECG data, EKG data, EMG data, galvanic skin response, or peripheral temperature. The neurological data obtained by the system may include, for example, one or more spectral power bands, absolute or relative EEG power, neurological connectivity, EEG delta data, EEG theta data, EEG alpha data, EEG SMR data, EEG beta data, task load metrics, or attention metrics.

According to some implementations, obtaining the physiological and neurological data may include measuring a stress response level and an arousal level in anticipation of a visual stimuli, and measuring a stress response level and a second arousal level responsive to a stress stimuli. In this regard, the stress stimuli may include an auditory or physical stimulus (e.g., a loud noise).

During each assessment exercise, raw EEG signals may be transferred over a wired connection or wirelessly (e.g., via Bluetooth) to the system (including, e.g., computing device 106 and/or server 112) for processing, recording and real-time analytic display. Accordingly, the following example assessment data may be collected:

TABLE E

| | DATA | DESCRIPTION | VISUALIZATION |
|---|---|---|---|
| 1 | Processed qEEG | Filtered mV activity from | Time series dB frequency of each |
| | | each of 19 EEG sensors. | signal. |
| 2 | Mirror Neuron | Cortical mV activity across | Real-time mapping of color- |
| | Network Activity | right and left hemispheres. | indicated MNN activity on multi- |
| | | | axis 3D volume, axial and sagittal |
| | | | views. |
| 3 | Mirror Neuron | mV connectivity between | Real-time mapping of color- |
| | Network | MNN nodes in each right and | indicated MNN output between |
| | Connectivity | left hemisphere. | each node on multi-axis 3D |
| | | | volume view. |
| 4 | Mirror Neuron | mV output from each MNN | Real-time spectral power output |
| | Network Output | node in each right and left | from each MNN node in polar |
| | | hemisphere. | view of left and right hemisphere. |
| 5 | Default Mode | Cortical mV activity across | Real-time mapping of color- |
| | Network Activity | right and left hemispheres. | indicated DMN activity on multi- |
| | | | axis 3D volume, axial and sagittal |
| | | | views. |
| 6 | Salience Network | Cortical mV activity across | Real-time mapping of color- |
| | Activity | right and left hemispheres. | indicated Salience Network |
| | | | activity on multi-axis 3D volume, |
| | | | axial and sagittal views. |
| 7 | Pre and Post | Hz frequency band power for | Mapping of color indicated SMR |
| | Motor Task SMR | activity for theta = 4.0-8 Hz, | state on multi-axis 3D volume, |
| | Cortex Activity | alpha = 8.0-12 Hz, SMR = | axial and sagittal views. |
| | | 13.0-15 Hz, beta1 = 15.0-20 | |
| | | Hz, and beta2 = 20.0-30 Hz. | |
| 8 | Pre and Post | Hz frequency band power for | Hz bar graph of SMR MNN ROI |
| | Motor Task SMR | SMR activity of MNN ROIs | power for each pre and post motor |
| | Asymmetry | in each right and left | task state. |
| | | hemisphere. | |
| 9 | Time Series SMR | Hz frequency band power for | Time series Hz plot of SMR MNN |
| | Asymmetry | SMR asymmetry of MNN | ROI asymmetry with exercise |
| | | ROIs throughout assessment | event markers and trend line. |
| | | exercise period. | |
| 10 | Pre and Post | Hz mean frequency band | Time series plot of dB output for |
| | Motor Task | power for pre and post motor | each channel in each right and left |
| | Analysis of all | imagery periods in each right | hemisphere. |
| | qEEG Channels | and left hemisphere. | |
| 11 | Attention Metric | Hz composite measure of | Time series Hz plot of Attention |
| | | attention-related bands | Metric with exercise event markers |
| | | throughout assessment | and trend line. |
| | | exercise period. | |
| 12 | Workload Metric | Hz composite measure of | Time series Hz plot of Workload |
| | | workload-related bands | Metric with exercise event markers |
| | | throughout assessment | and trend line. |
| | | exercise period. | |

The system obtains a baseline data threshold for the obtained first physiological and neurological data (304). The initial neuro/physical assessment data may be used to create a baseline from which athletes can be further evaluated and trained to prevent, and as necessary, recover from lower body injuries before return to play. These injuries include hamstring strains, lateral ankle sprains, adductor strains, high ankle sprains, and medial collateral ligament tears among others. The subject technology has been found to counter the devastating effect of ACL (Anterior Cruciate Ligament) tears, which frequently inhibit athletes' from continued team participation. In some implementations, the baseline data threshold includes a reaction time to the stimulation (e.g., to the visual or auditory stimulation).

According to some implementations, the baseline data threshold may include a baseline qEEG recording with eyes closed, open and then visual arrest. The qEEG recording may be followed by segments consisting of cognitive exercises, mental imagery motor tasks further followed by physical motor tasks. In some implementations, the baseline data threshold represents a threshold range corresponding to normal athletic conditions for the particular athlete being rehabilitated. In some implementations, the threshold range pertains to performance metrics prior to the injury being rehabilitated. In this regard, the system may obtain the first physiological and neurological data prior to the person experiencing an injury so that the system can then quantify, based on subsequent assessments, improvement in athletic performance and rehabilitation towards pre-injury conditions.

In some implementations, the baseline data threshold may be based on training data collected from analysis of a population of tests or test subjects (e.g., athletes). For example, the system may include a machine learning algorithm and/or implement a neural network that collects neurological and physiological data for the population. The algorithm may determine baseline ranges of values for the data, for a particular demographic or for information pertaining to a given athlete profile. Machine learning may include models, equations, artificial neural networks, recurrent neural networks, convolutional neural networks, decision trees, or other machine readable artificial intelligence structure.

The system then selects a second predetermined visual stimulation based on the obtained physiological and neurological data and the baseline data threshold (306). The visual stimulation may be configured to prompt a neurological metric toward the baseline data threshold. According to various implementations, the neurological metric is indicative of connectivity, activation, or symmetry measured in the one or more target regions of the athlete's brain. In this regard, the system determines, based on the neurological data measurements, a level of connectivity, activation, or symmetry in the regions of interest.

One or more task segments may be generated for the athlete, with each segment associated with the one or more actions (e.g., physical or mental tasks). The visual stimulation may include prompting the athlete to perform the action(s), and the second physiological and neurological data may be obtained in real-time responsive to a performance of at least one of the physical or mental tasks by the athlete.

According to various implementations, the biometric interface 200 is configured to obtain and render electrical activity occurring in different sections of the brain during the one or more actions. The subject technology comprises software that analyzes the physiological and neurological conditions to make determinations as to how much attention was given to the activity (e.g., by way of electrical power measured from the brain). The software may then adjust the particular task segments provided to the athlete for a session based on the analysis and select or reselect visual stimulation based on the analysis. For example, the system may change a speed at which a task is to be performed or given, or a speed at which changing tasks occur over time, and monitor the result.

In some implementations, each task segment may include one or more algorithms which process the collected data obtained from sensors 102 (including, e.g., EEG data) in real time. The algorithms may be configured to make determinations as to whether additional tasks should be generated, or currently performed tasks should be altered, before the next predetermined task in the list is performed. Real time decisions with regard to the obtained data may be based on predetermined programming or based on machine learning. In this manner, the system may adapt in real time to meet individual needs of the athlete being rehabilitated and/or to provide further tasks more suitable for rehabilitating the athlete.

For example, during rehabilitation, the system may detect a real time reduction in the subject athlete's performance based on the real-time data. For example, the obtained data may be indicative of negative progress towards rehabilitation of a specific injury. The system may identify the issue and adapt in real time to prevent further injury to the athlete. The audible or visual stimulation provided to the athlete may be altered, for example, by selection of different visual queues or instructions, or changing the speed of the queues or instructions provided to the athlete. Additionally or in the alternative, task may be altered to, for example, reduce repetitions and/or length of stress on the athlete, and/or the rehabilitation equipment may automatically be adjusted electronically for less resistance during a particular exercise.

The system obtains, after the second visual stimulation is selected, second physiological and neurological data pertaining to one or more actions of the person responsive to the altered visual stimulation (308). This second physiological and neurological data may be obtained after collection of the initial assessment data. As described previously, the data may be obtained by way of the sensors 102 using biometric interface 200. The athlete may be prompted to perform one or more physical or mental tasks, and the second physiological and neurological data may be obtained in real-time responsive to a performance of at least one physical or mental tasks by the athlete.

Based on the collected data, the system then determines a deviation between the baseline data threshold and the second physiological and neurological data (310). In some implementations, determining the deviation includes determining a magnitude of deviation between the first reaction time and a second reaction time to the selected visual stimulation. In some implementations, the deviation may be represented by data modeling or machine learning. In this regard, the system may quantify the deviation based on a given population of athletes and the athletes' progression toward rehabilitation of a similar injury. This deviation may further be represented in real time within athlete monitor section 226 of the biometric interface 200 (e.g., by updating a moving object toward a target), and/or provided for display to the athlete (e.g., by display device 108).

In some implementations, as shown in FIG. 3, the system may be instructed (e.g., by the clinician) to continue an assessment unless the data falls within a certain tolerance or range (e.g., within a range of “normal”). In this regard, the athlete may continue rehabilitation towards a goal represented by the collected data and the baseline.

In the depicted example, when a certain tolerance is reached, or upon selection by the clinician, the system further generates a neurological and physiological protocol based on the deviation (312). In this regard, a snapshot of the neuro-physiological status of the athlete may be displayed at any given time, in addition to a representation of the athlete's progress to a given goal (e.g., pre-injury performance for a particular muscle or muscle group). The system may then auto-generate a training protocol that provides specific exercises that may be performed by the athlete to maintain status quo (e.g., after rehabilitation) or to progress further to the rehabilitation goal.

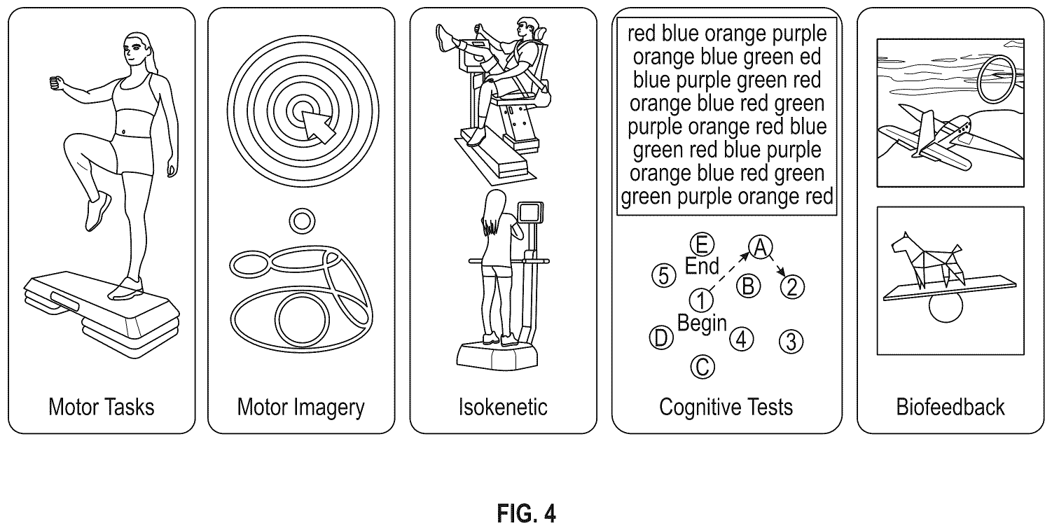

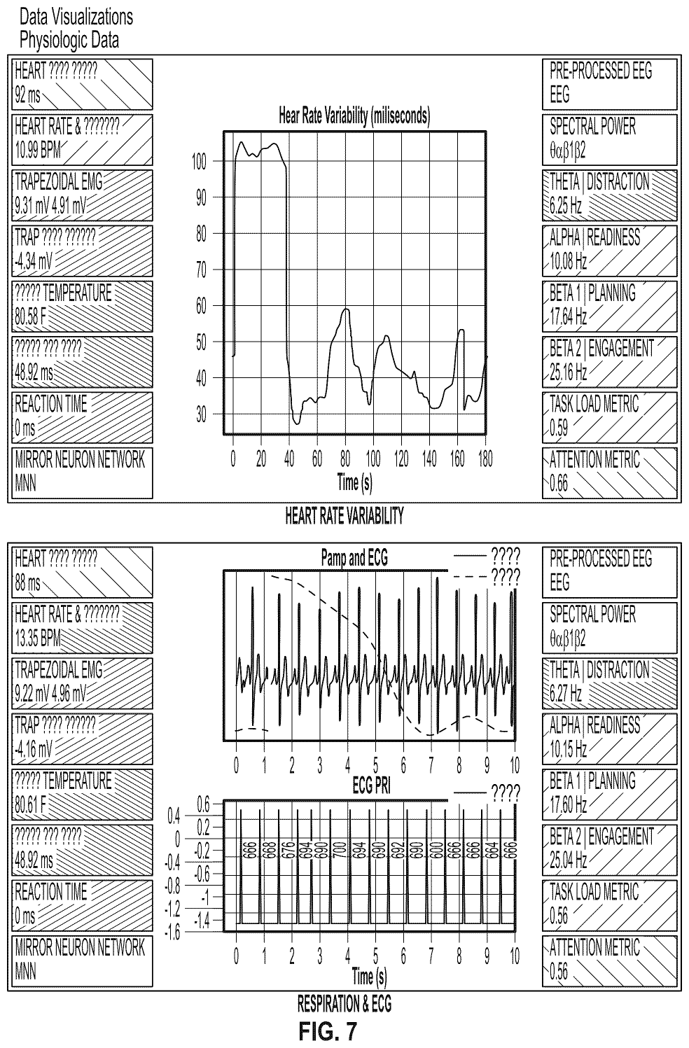

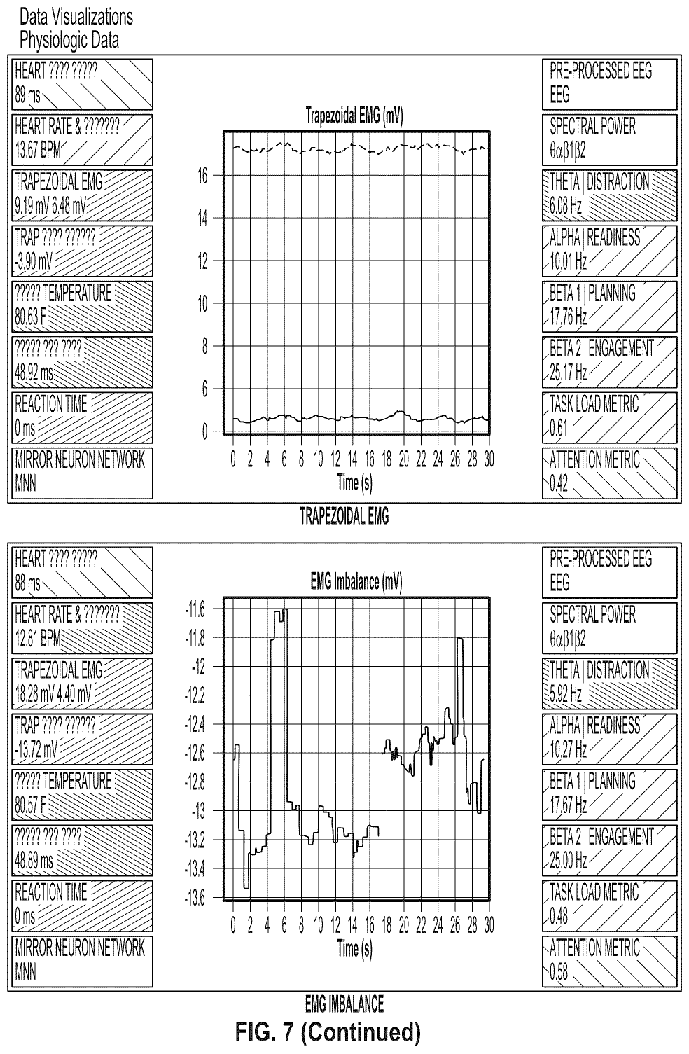

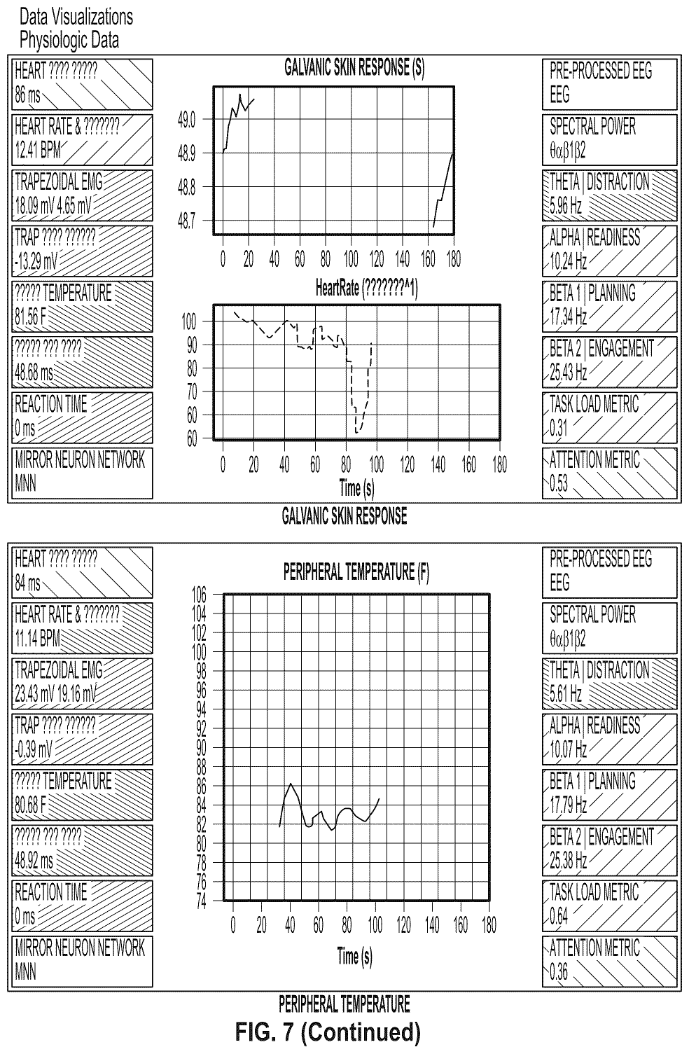

At any stage of the rehabilitation process, the obtained physiological and neurological data may be used to generate comprehensive baseline assessment report for each athlete. For example, after an initial assessment (e.g., analysis of the first physiological and neurological data), the report may provide a baseline neurological and physiological (or physical) status of the athlete, the basis to formulate particular performance, injury prevention and rehabilitation protocols, a reference from which to measure neuro-physical progress during subsequent assessment and training sessions. This data may collectively, for a population of athletes, be used as a source with which to build an aggregated athletic lifecycle database (see, e.g., FIG. 4). According to various implementations, various reports and associated metrics may be generated and provided by the system, either in real time during an assessment and rehabilitation session, or to provide results of the session or an athlete's rehabilitation status or progress. Example reports and reporting metrics are shown in FIGS. 7A through 7Z.

FIG. 3B depicts a second example process 350 for physiological and neurological rehabilitation of an athlete, according to aspects of the subject technology. For explanatory purposes, the various blocks of example process 350 are described herein with reference to FIGS. 1, 2A, and 2B, 3A, and 4-9 and the components and/or processes described herein. The one or more of the blocks of process 350 may be implemented, for example, by a computing device, including a processor and other components utilized by the device. In some implementations, one or more of the blocks may be implemented apart from other blocks, and by one or more different processors or devices. Further for explanatory purposes, the blocks of example process 350 are described as occurring in serial, or linearly. However, multiple blocks of example process 350 may occur in parallel. In addition, the blocks of example process 350 need not be performed in the order shown and/or one or more of the blocks of example process 350 need not be performed.

The system receives an injury type pertaining to a physical injury experienced by a patient (352). According to the depicted example, a clinician authenticates to computing device 106 or server 112 and initiates the biometric interface 200 to begin a rehabilitation session for a patient. The clinician may enter patient data through the interface or select the patient, as previously described. The clinician may then select an injury type to be rehabilitated.

The system then selects, based on an injury type, one or more target regions of the patient's brain for assessment of a neurological metric associated with a neurological network of the patient's brain (354). As described previously, the neurological network may include a Mirror Neuron Network, Salience Network, or Default Network of the patient's brain. The selection may occur automatically based on indexing the database 110 by injury type. In some implementations, the indexing may include patient demographic, age, and certain physiological characteristics (e.g., weight, height, etc). In some implementations, the selection may be made by the clinician using a selection menu provided by the biometric interface 200.

One or more neurological sensors are provided and/or activated for measuring neurological activity within the one or more target regions of the patient's brain (356). As described previously, the sensors 102 may include EEG sensor(s), ECG sensor(s), EMG sensor(s), ACCL sensor(s), a respiration monitor, heart monitor, galvanic skin response device or electrodes, oxygen monitor, and/or a peripheral temperature sensor. The sensor(s) 102 may embodied in, for example, one or more wearables such as a watch, chest or arm or thigh band, head unit, glasses, or combination thereof.

Once the sensors are set to collect data, the system provides, via a display screen associate with computing device 106 or server 112, a first predetermined visual stimulation (358). For the purposes of this disclosure, visual stimulation includes directives, output, visual stimuli, as well as instructions to undertake a physical or mental task. The visual stimulation may include a prompt to perform a physical or mental task (see, e.g., Tables A, B, C, D). The task may be selected automatically based on the injury type and/or the region of the brain corresponding to the injury. For example, the database 110 may include physical and/or mental tasks that are predetermined for adjusting a neurological metric toward or away from a baseline (in addition to, e.g., physically rehabilitating the injury). The database may be indexed by the application 200 based on the injury type and/or region of the brain and/or the neurological metric (activation, connectivity, symmetry, etc.) to identify the physical or mental task to be applied in the patient's rehabilitation protocol, and may direct visual stimulation to cause the patient to perform the task.

The system then initiates athletic performance of the patient based on the first predetermined visual stimulation (360). In this regard, the patient performance may be initiated by way of the stimulation. For example, if the stimulation is a pacer exercise ex, the system may monitor the patient while the patient participates in the exercise, adjusting the stimuli of the exercise to move the patient's brain activity for the neurological metric toward the baseline goal. In some implementations, the visual stimulus may include a virtual graphic on the display 108 that is adjusted by way of the patient changing the patient's brain and/or physiological activity, as measured by patient sensors 102.

With reference to FIG. 1, the neurological sensors 102, computing device 102, and stimulus 108 provides a feedback loop to retrain the target regions of the brain, thereby moving those regions towards the predetermined baseline and facilitating the rehabilitation of the patient. In this regard, the system obtains, via the neurological sensor(s) 102, first neurological data pertaining to the athletic performance of the patient reacting to the first predetermined visual stimulation (362), and the system compares the obtained first neurological data to the baseline data threshold (364). As described previously, the first neurological data comprises measurements pertaining to the neurological metric (activation, connectivity, symmetry, etc.) for the target region(s).

In the depicted example, a baseline data threshold for the neurological metric is determined. In some implementations, the baseline may be predetermined based on a given patient population. For example, neurological training data pertaining to the athletic performance of a non-injured patient population reacting to the first predetermined visual stimulation may be obtained overtime and stored in the central database 110. This training data for example, may include and pertain to the given neurological metric in a stable state, and the baseline data threshold for the neurological metric may be determined based on the training data.

In some implementations, the system may be used to evaluate and rehabilitate existing patients for which a pre-injury assessment has been made. In this regard, the first predetermined visual stimulation may be part of an overall examination of the patient prior to injury. For example, an athlete may be evaluated prior to being admitted to an athletic program or team. The system may be used to conduct qualitative analysis of the athlete's brain prior to play. In this regard, each network may be evaluated for each neurological metric (activation, connectivity, symmetry), thereby providing an individualized baseline for all metrics and networks and regions, pre-injury. When an athlete becomes injured, the system may be used to assess and rehabilitate the athlete in accordance with the following steps.

In some implementations, the first visual stimulation may be stimulation that is applied as part of a series of visual stimulations in a rehabilitation protocol (see, e.g., Tables A, B, C, D). For example, on entry of the injury and patient information, the biometric interface 200 may load a rehabilitation protocol including a series of tasks. The visual stimulation may prompt the patient to perform the task, or may prompt a clinician to guide the patient in performing the task.

The system selects a second predetermined visual stimulation configured to prompt the neurological metric toward the baseline data threshold (368), and obtains, via the one or more neurological sensors 102, during the second predetermined visual stimulation, second neurological data pertaining to one or more actions of the patient responsive to the second predetermined visual stimulation (370). As described previously, the second visual stimulation may include visual stimulation that is applied after the baseline is set. In some implementations, the second visual stimulation may be stimulation that is subsequently applied in a series of visual stimulations.

The system determines, based on the deviation, a state of the neurological metric indicating whether the neurological metric adjusted toward the baseline threshold (372). For example, if the system is measuring activation, the system may calculate a level of spectral power in certain frequency ranges for the region of the brain associated with the injury. The baseline threshold may include a predetermined power level, which the calculated level is compared. If the calculated level, obtained after the second visual stimulation, moved towards the baseline level as compared with the first visual stimulation (or prior stimulation in a series of stimulations) then the state may indicate that the neurological metric (activation) has adjusted toward the baseline threshold.

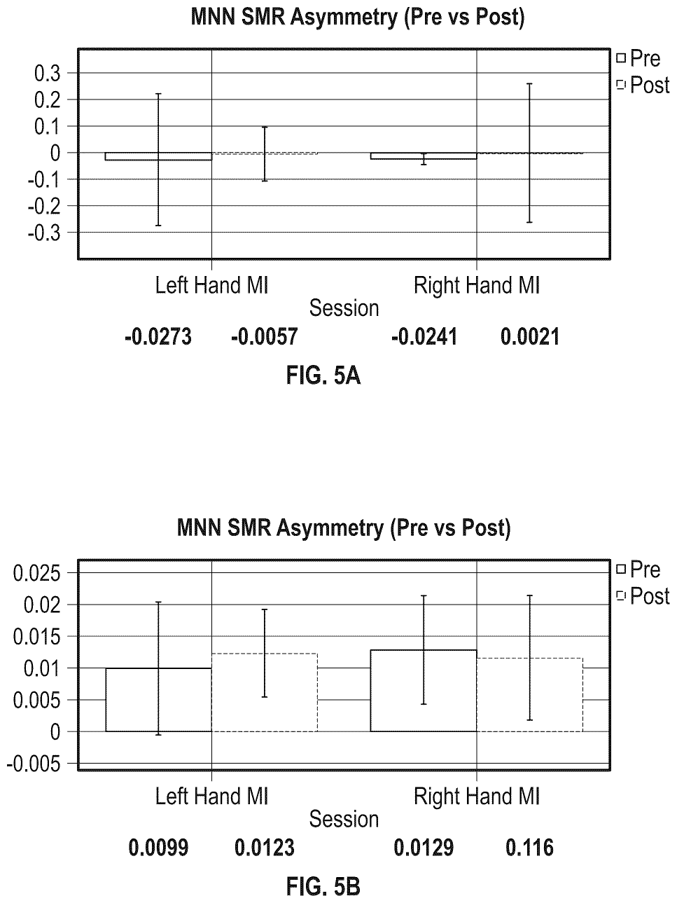

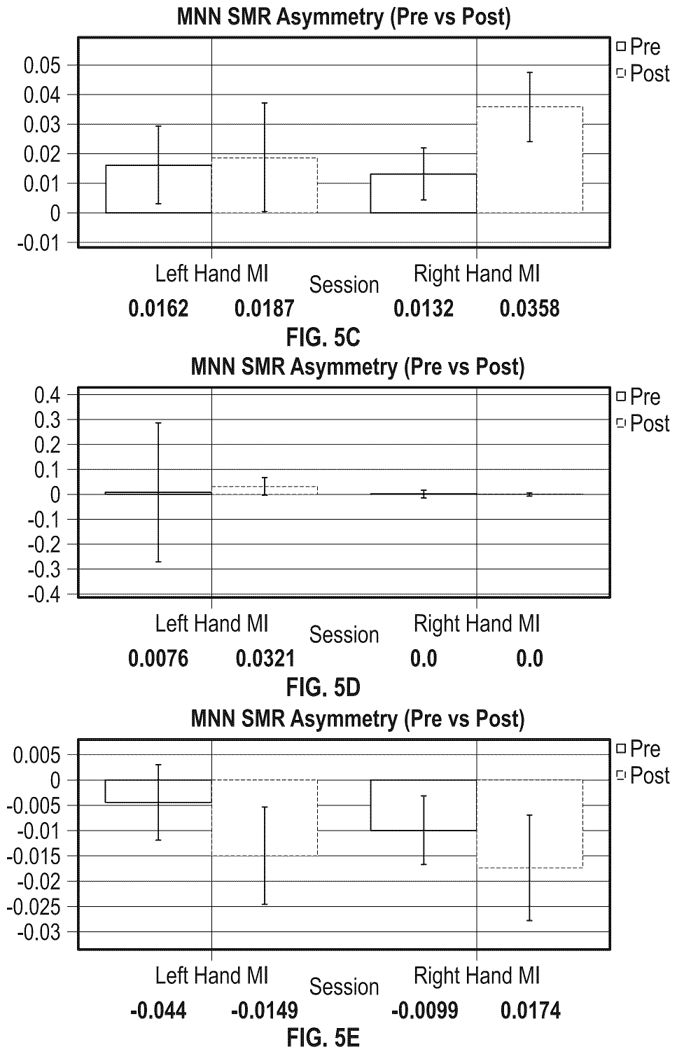

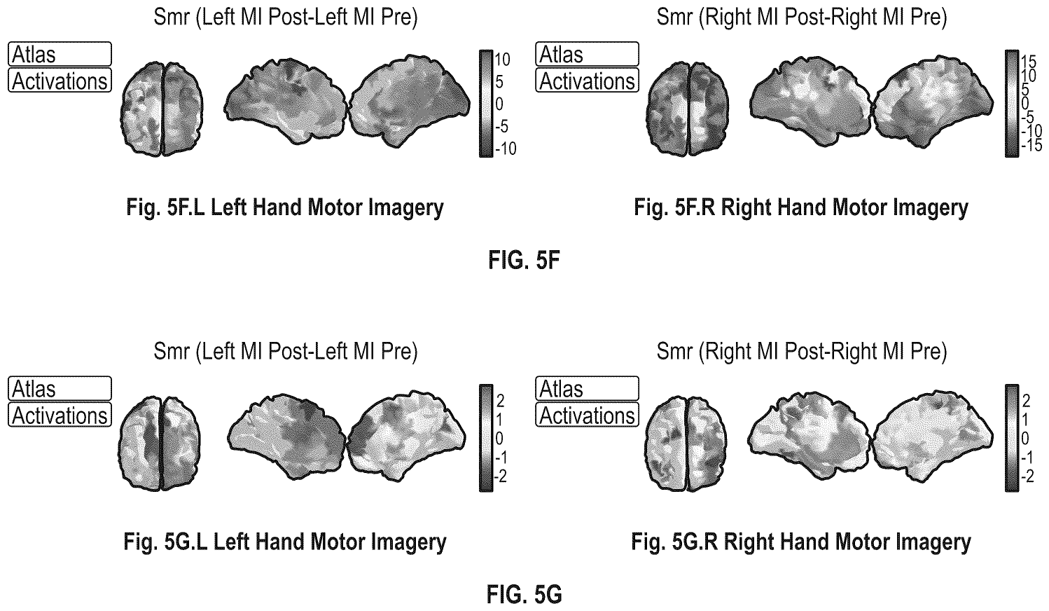

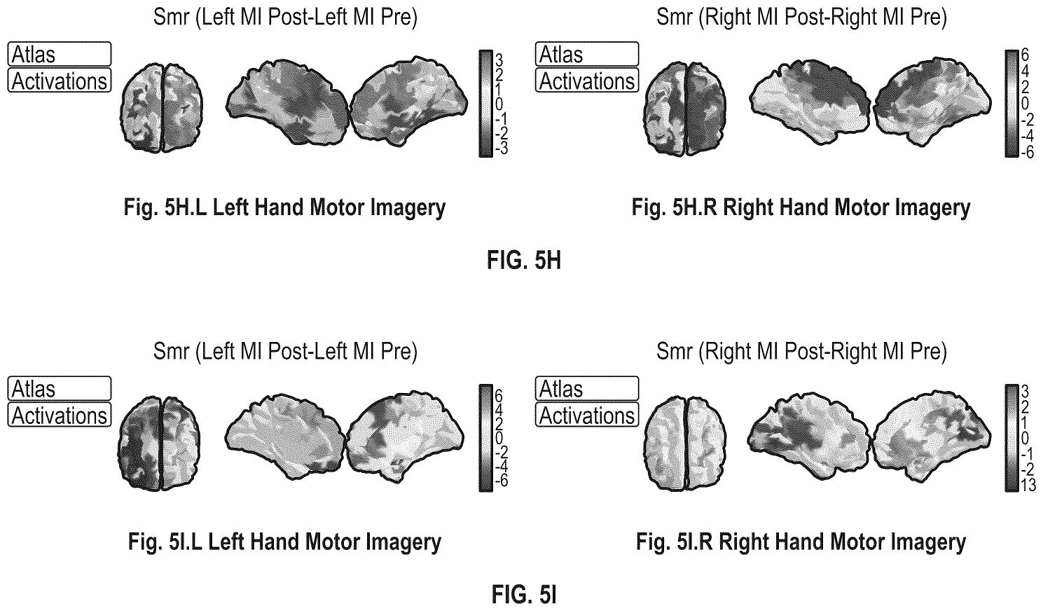

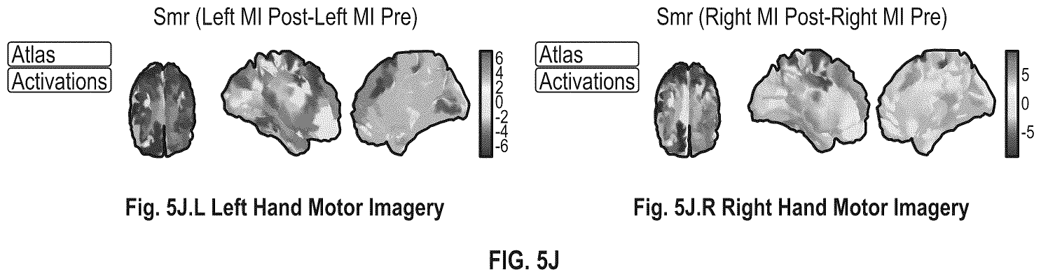

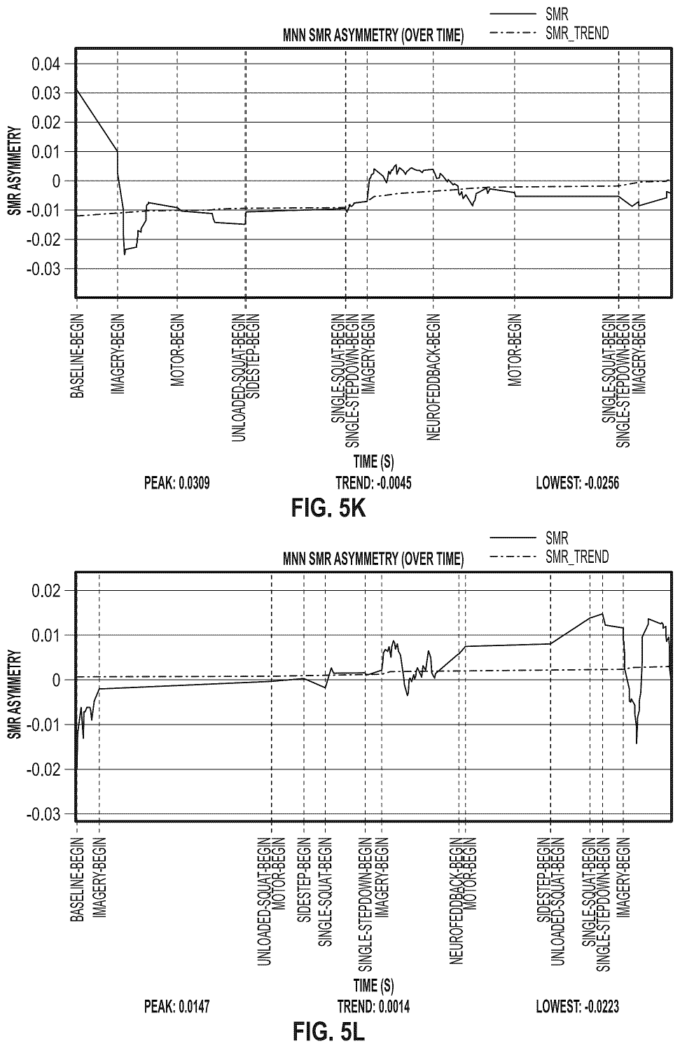

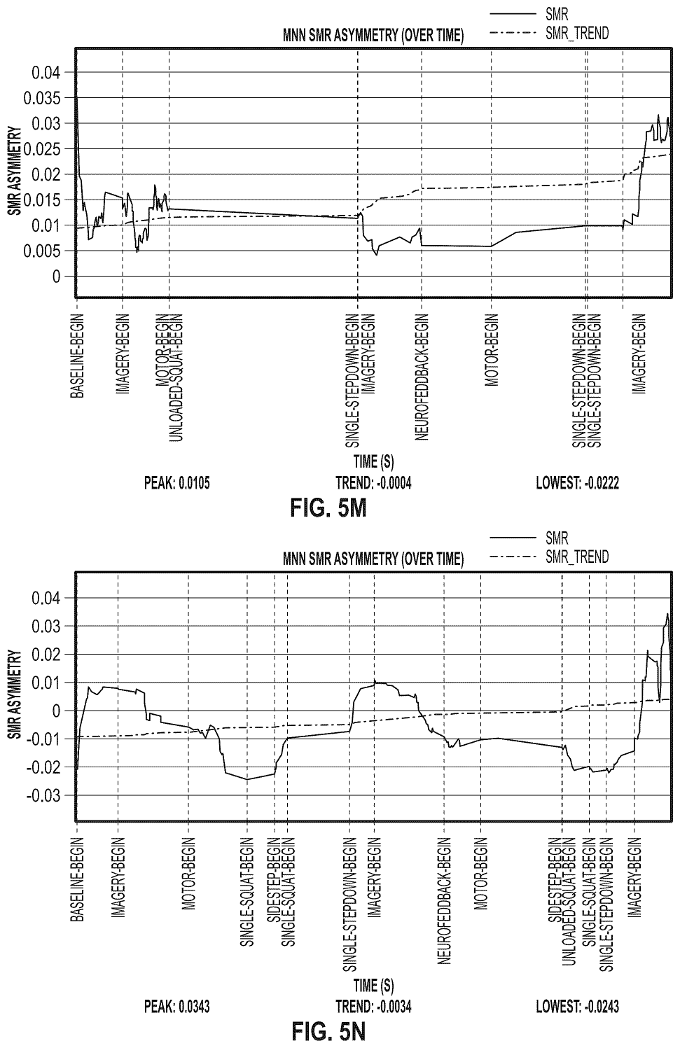

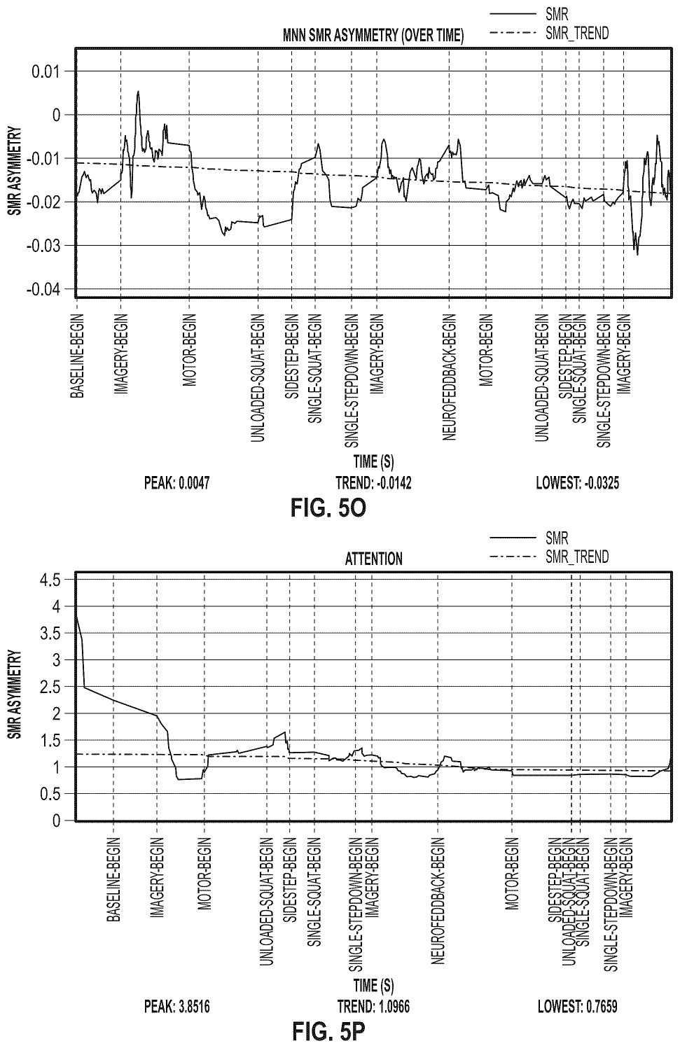

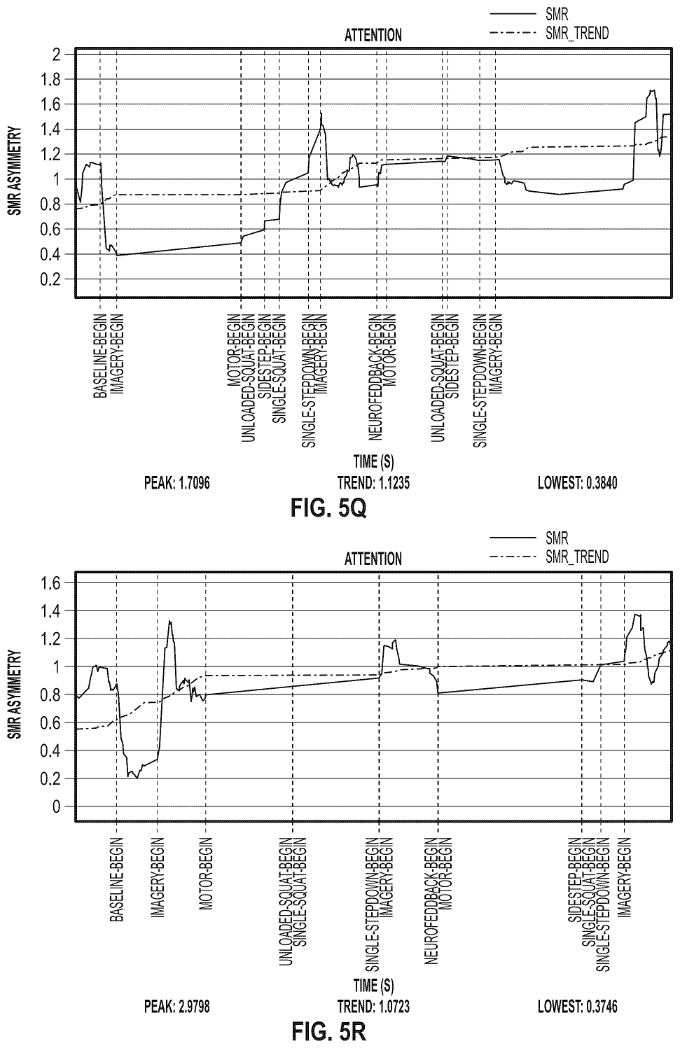

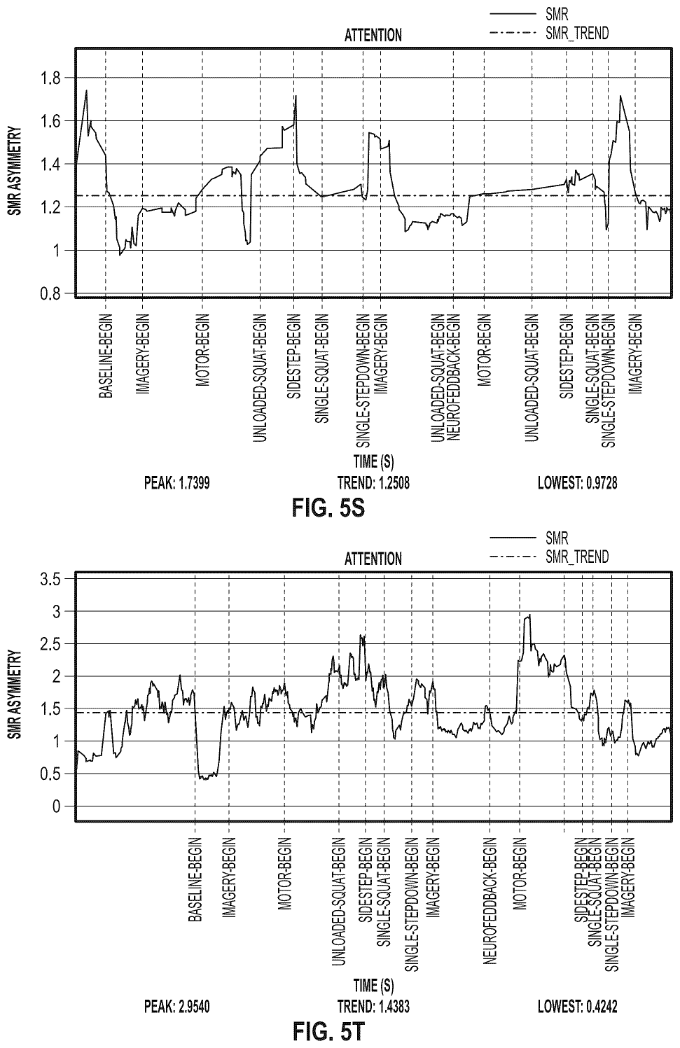

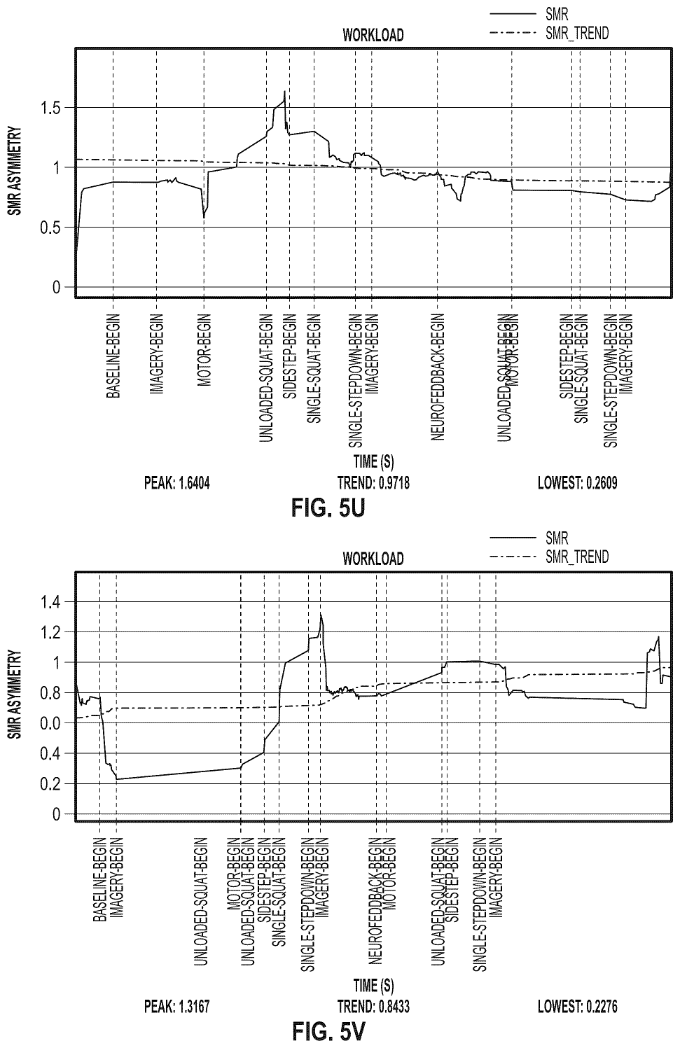

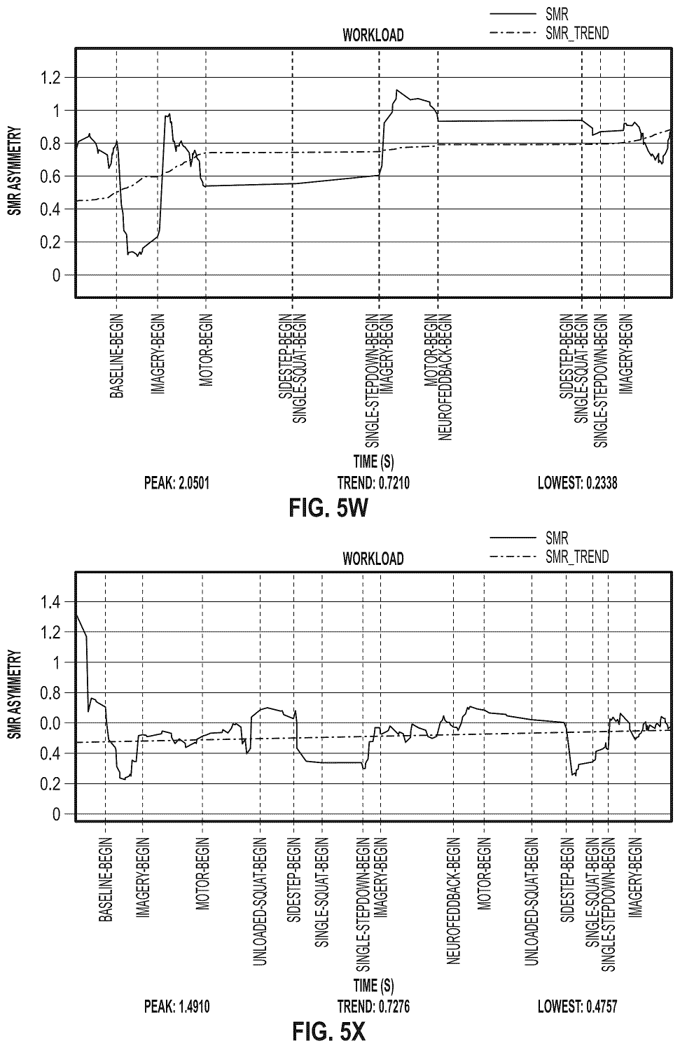

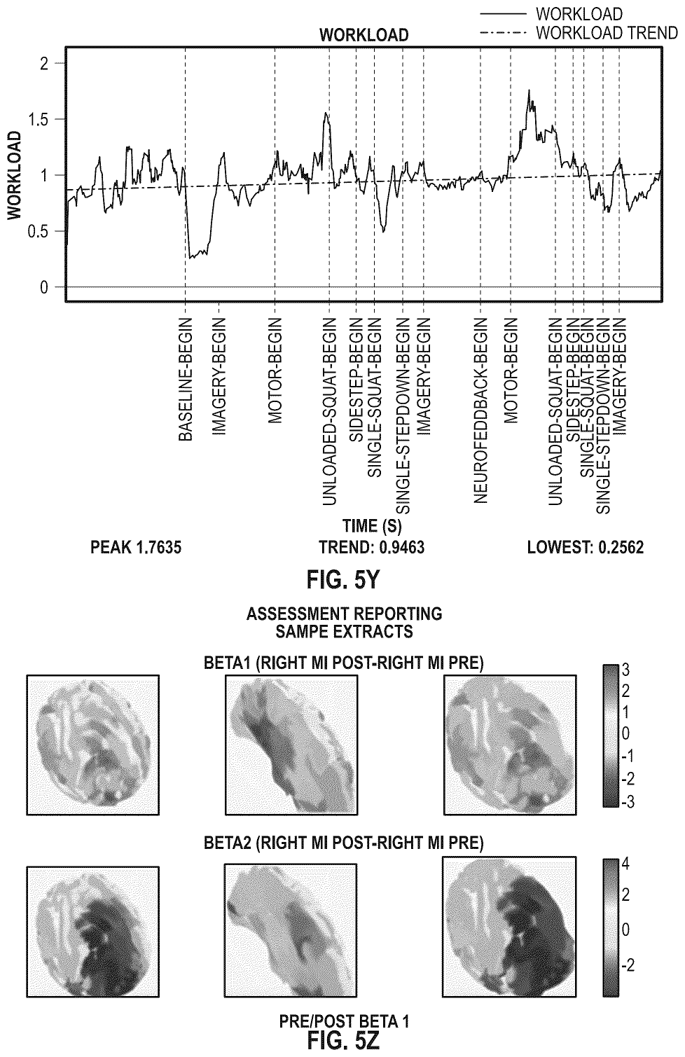

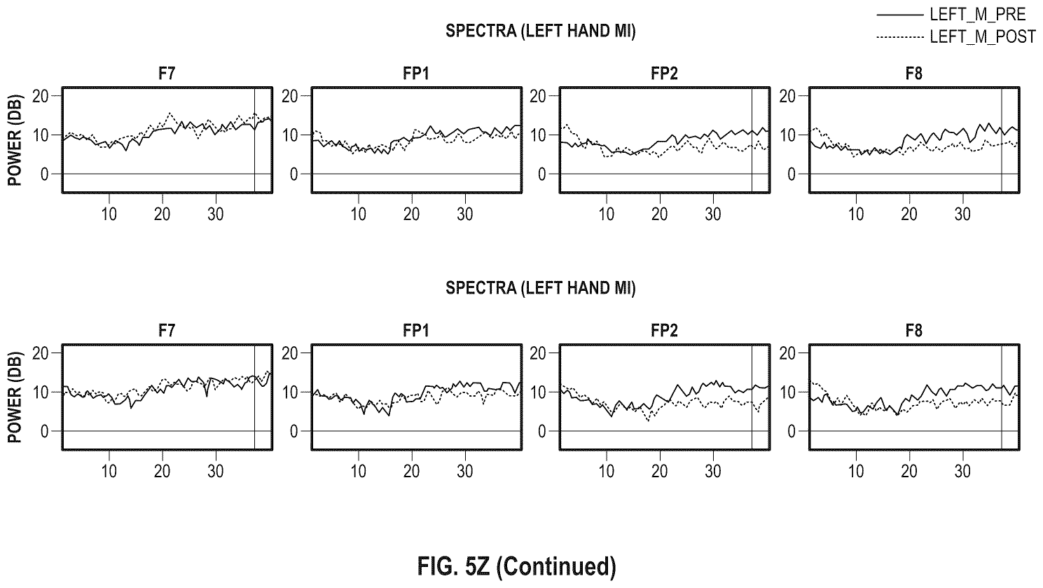

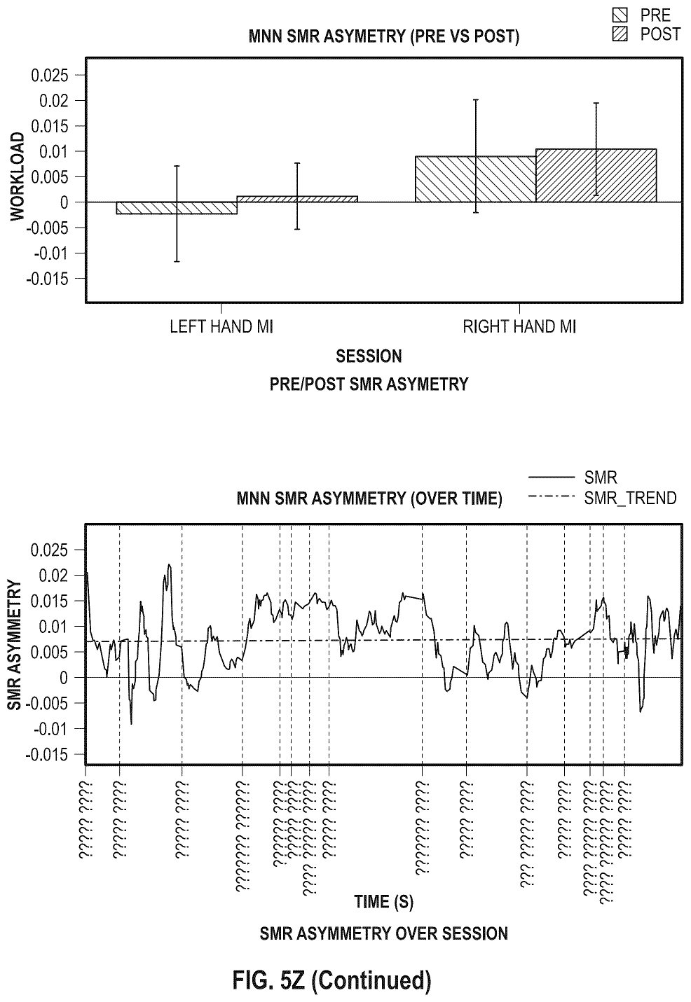

After each iteration of stimuli, or upon completing a rehabilitation session, the system provide, on a display screen associated with computing device 106 and/or server 112, an indication of the state of the neurological metric and a graphical representation of the second neurological data and the deviation. FIGS. 5-7 provide various examples of graphical representations of neurological data which may be displayed within the biometric interface 200 (e.g., in interface display areas 210, 212, 214 and/or 217).

As described previously with regard to FIG. 3A, physiological data (e.g., heart rate, heart rate variability, SpO2, respiration rate, ECG/EKG data, EMG data, etc.) may also be collected and the patient may be provided the respective visual stimulation to move the patient to an overall rehabilitated state. Additionally or in the alternative, some implementations may set a baseline for the physiological data, select stimuli for rehabilitating the patient with respect to an injury through physical and/or mental tasks that rehabilitate one or more physiological parameters toward their respective baselines, as previously described with regard to neurological data.

Accordingly, the disclosed system of rehabilitation includes neuro-feedback exercises, physical exercises, and/or inter-active exercises. For example, the system may identify that a person is exhibiting a tremendous amount of stress based on analysis of physiologic data, such as trapezoidal EMG and respiration. A technician does not need to know how to analyze this data. The system will deliver and/or guide the modalities based on the trained model. For example, the system may instruct delivery of a breathing exercise or a balance exercise. The system may guide the patient to perform an exercise, for example, on a rehabilitation device 104 (e.g., resistance training on a strength machine). The system may collect data during the exercise to slow or speed up the exercise. The system may deliver a virtual pacer exercise where a graphic is shown a screen and regulate the patient's breath based on adjusting the graphic on the screen. The system may continue to guide each modality to adjust/correct the rehabilitation metrics.

In some implementations, psychometrics may be added to observe confidence as an input. For example, a risk propensity index value may be obtained based on a test. The test may then be repeated after a training session to determine whether confidence is returning to pre-injury levels, or to levels normally seen in a population.

As described previously, process 350 or portions thereof may be conducted by a machine learning algorithm and/or a neural network that makes decisions based on neurological and physiological data of a predetermined population. For example, the algorithm may determine baseline values/ranges of values and/or make selections (e.g., neurological regions, neurological metrics and stimulations) based on information pertaining to injury type, a given patient profile (e.g., age, height, weight, BMI, and/or demographic, etc.) and/or physiological data. The system may select the target regions of the patient's brain for assessment based on injury type, and select a stimulation (physical or mental task) to be performed based on the patient's profile, injury, and current level of rehabilitation. Machine learning may include models, equations, artificial neural networks, recurrent neural networks, convolutional neural networks, decision trees, or other machine readable artificial intelligence structure.

Working with the data collected during the baseline assessment process, the subject technology thus facilitates development of training exercises to be performed during specially scheduled sessions and during physical therapies. Neuro-physical data may be collected during each training exercise, and data may be analyzed to determine the efficacy of each prescribed training protocol. Data and outcomes may be uploaded and/or stored in database 110 to supplement existing populations and training data. Live analytics are provided via the biometric interface 200 so that a trainer may observe a phenomenon. The trainer may then try to replicate the phenomenon by replaying the modality (e.g., have the patient perform another step down exercise). Deficiencies may be identified in reports that show more data over time.

Throughout the procedure, the system may determine a level of neuroplasticity for each of the evaluated regions. For example, after a baseline is taken, the patient's neuroplasticity level for activation may be set to a give value (e.g., 3 on a scale of 1-10, with 10 being considered stable in a given population). The level may be associated with the injury and other patient characteristics. In this regard, as the patient is rehabilitated, this value may change and the new value used to assess improvement over time. In some implementations, the value may be compared to a second threshold (e.g., below baseline) to assess whether the patient may resume normal activity after injury. For example, a athletic institution may set the second threshold to a certain value (e.g., 7) that is indicative of return to play. If the athlete meets the value then the athlete may resume athletic activities. If the athlete is unable to meet the value then the athlete may be prevented from returning to play until the value is met.

The analysis and data provided by the subject technology, may facilitate determination of an athletes beginning physical, neurological, psychological and practical predisposition to injury. The system further develops specific neuro/physical training and assessment routines to minimize known risks, assesses athletes' post-injury/pre-rehabilitation neuro/physical status, prepares and implements corresponding neuro/physical rehabilitation training and assessment plans, measure athletes' readiness for a safe and successful return-to-play, monitor athletes' continuing performance to avoid risk of re-injury. Over time, this neuro/physical data will form the basis by which to improve various assessment and training protocols, and guidelines for return-to-play. Results of the system described herein may also provide the foundation for publishing clinical research on various neuro/physical initiatives.

Many aspects of the above-described example processes 300 and 350, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

FIG. 4 depicts example physical and mental tasks utilized by the subject technology to induce and measure a neuro-physiological response, according to aspects of the subject technology. The previously described visual stimulation may be provided to facilitate one or more physical and/or mental tasks which are performed while neurological and physiological data is collected. Physical tasks include, for example, motor tasks such as stepping or weight training or running or isokinetic tasks. Mental tasks include, for example, motor imagery, cognitive tests, and biofeedback (e.g., pacer exercises). For example, in some implementations the patient may be fitted with a EEG headset and other sensors 102 previously described, and data collected in real time as the patient performs the tasks. In all implementations, any of the sensors 102 (e.g., EEG headset, ECG, etc.) may include wireless transmission of data or local storage to enable free movement of the patient while data is collected. For example, physical exercises may include walking over obstacles or throwing a ball.

FIGS. 5A through 6Z depict example reports and associated metrics provided by the disclosed system. FIGS. 5A through 5J illustrate results of motor tasks. Data in FIG. 5A, for Patient 1, illustrates averaged SMR (13-15 Hz) Asymmetry for the Mirror Neuron Network ROIs from the motor imagery periods before and after the motor training tasks. Asymmetry Score=(Left−Right)/(Left+Right). The patient had surgery on his right ACL after high school injury. His asymmetry for the Mirror Neuron Network Regions of Interest remained unchanged with both his left and right hand pre and post motor imagery after completing the motor training tasks. His right-hand asymmetry slightly decreased pre and post motor imagery, indicating that his MNN ROI remained consistent during both motor training tasks.

In FIG. 5B, Patient 2 suffered right knee ACL. He has improved Asymmetry for the Mirror Neuron Network Regions of Interest with his left hand pre and post motor imagery after completing the motor training tasks. He made the greatest improvement in his left hand in his second post motor Imagery, indicating that his MNN ROI did improve after the motor training tasks.

In FIG. 5C, Patient 3 had 2 bilateral Achilles' injuries in successive years and is reported fully rehabilitated. He has improved Asymmetry for the Mirror Neuron Network Regions of Interest with his right hand pre and post motor imagery after completing the motor training tasks. His left hand decreased slightly. He made the greatest improvement in his right hand in his second post motor Imagery, indicating that his MNN ROI did improve after the motor training tasks. In FIG. 5D, Patient 4 was 12 weeks post right ACL surgery. The patient exhibited moderate change of Asymmetry for the Mirror Neuron Network Regions of Interest only in his left hand after completing the initial motor training task. There was negative change after his second post motor Imagery, indicating that his MNN ROI did not improve after the initial motor training tasks.

In FIG. 5E, Patient 5's injury was right ACL surgery. His Asymmetry decreased for the Mirror Neuron Network Regions of Interest with both his right and left hand pre and post motor imagery after completing the motor training tasks. His asymmetry decreased less in the left hand than in the right hand after completing the motor imagery tasks.

FIG. 5F illustrates cortex activity, pre and post motor task for Patient 1. Visualizations illustrate the difference in Pre and Post Motor Imagery Task frequency band powers as T-scores (masked for significant values only) computed for all ROIs, by each hand, and mapped onto a 3D cortex. Positive values represent Post>Pre. The leftmost column shows the entire cortex, while the two columns on the right show the two hemispheres of the cortex. The patient's 3D Cortex shows 10 degrees of freedom in left SMR pre/post motor imagery and 15 degrees of freedom in the right SMR pre/post motor imagery. All positive values represent the post motor imagery compared to the premotor imagery. We show the full cortex as indicated on the left and the other 2 pictures show the two hemispheres of the cortex. As you can see from the key on the right the normal areas are in shades of green, where consistent activity is in yellow, orange and red. The MNN was activated consistently in pre/post motor imagery tasks indicating the preparation (readiness potential) and initiation (motor potential) of the movements were activated in the MNN Network. The MNN was active consistently in post motor imagery tasks. Reference of Degrees of Freedom illustrate the number of values in the final calculation of a statistic that is free to vary or in otherwards, the number of independent ways by which a dynamic system can move.

In FIG. 5G, Patient 2's 3D Cortex shows the most consistent degrees of freedom (2) of all the athletes for the SMR pre/post motor imagery. All positive values represent the post motor imagery compared to the premotor imagery. We show the full cortex as indicated on the left and the other 2 pictures show the two hemispheres of the cortex. As you can see from the key on the right the normal areas are in shades of green, where consistent activity is in yellow; orange and red. The MNN was hyperactive and indicated by the red areas and was activated consistently in post motor imagery tasks indicating the preparation (readiness potential) and initiation (motor potential) of the movements were activated in the MNN Network. The MNN was consistently activated in post motor imagery tasks.

In FIG. 5H, Patient 3's 3D Cortex reflect 3 degrees of freedom in the left SMR pre/post motor imagery and 6 degrees in the right SMR pre/post motor injury. All positive values represent the post motor imagery compared to the premotor imagery. We show the full cortex as indicated on the left and the other 2 pictures show the two hemispheres of the cortex. As you can see from the key on the right the normal areas are in shades of blue/green, where consistent activity is in yellow, orange and red. The MNN is highlighted by the red areas and was activated consistently in post motor imagery tasks indicating the preparation (readiness potential) and initiation (motor potential) of the movements were activated in the MNN Network. The MNN was consistently activated in post motor imagery tasks.

In FIG. 5I, Patient 4's 3D Cortex shows 6 degrees of freedom on the left SMR pre/post motor imagery and 3 degrees of freedom on the right SMR pre/post motor imagery. All positive values represent the post motor imagery compared to the premotor imagery. We show the full cortex as indicated on the left and the other 2 pictures show the two hemispheres of the cortex. As you can see from the key on the right the normal areas are in shades of green, where consistent activity is in yellow, orange and red. The MNN was hypoactive and less activated in pre/post motor imagery tasks indicating the lack of preparation (readiness potential) and initiation (motor potential) of the movements in the MNN Network. A portion of the MNN (ie., left frontal cortex) was activated in post motor imagery tasks.

In FIG. 5J, Patient 5's 3D Cortex shows 6 degrees of freedom on the left SMR pre/post motor imagery and 5 degrees of freedom on the right SMR pre/post motor imagery. All positive values represent the post motor imagery compared to the premotor imagery. We show the full cortex as indicated on the left and the other 2 pictures show the two hemispheres of the cortex. As you can see from the key on the right the normal areas are in shades of green, where consistent activity is in yellow, orange and red. The MNN was significantly hypoactive and less activated in pre/post motor imagery tasks indicating the lack of preparation (readiness potential) and initiation (motor potential) of the movements in the MNN Network.

FIGS. 5K through 5N depict a time series for SMR Asymmetry. The depicted data illustrates the average SMR (13-15 Hz) Asymmetry for the Mirror Neuron Network ROIs averaged together over time. Asymmetry Score=(Left−Right)/(Left+Right).

In FIG. 5K, Patient 1's MNN SMR Asymmetry Trend began at −0.01 and progressed to 0 during the session. His MNN SMR Asymmetry trend line steadily increased as he progressed through the assessment. His MNN SMR activity increased above 0 during the first post mental imagery. In FIG. 5L, Patient 2's MNN SMR Asymmetry trend line was the only athlete that started above 0 and steadily increased throughout the training session. His MNN SMR activity increased during the second imagery segment and steadily increased throughout the second set of motor activities. In FIG. 5M, Patient 3's MNN SMR Asymmetry was the only athlete other than CM that started above 0 and steadily increased throughout the training session.

In FIG. 5N, Patient 4's MNN SMR Asymmetry although trending higher throughout assessment, his asymmetry trended downward beginning at the initial motor imagery and motor activities. SMR increased to 0.01 at the beginning of the post imagery but again continued downward thereafter. His SMR peaked briefly at the end of the assessment when he knew that the assessment was almost completed. In FIG. 5O, Patient 5's MNN SMR Asymmetry was the only athlete that started below 0 and steadily decreased throughout the entire training session. His MNN SMR Asymmetry activity increased during the first motor imagery and although briefly spiked above the trend line for the motor activity and the 2nd post motor imagery, there remained a steady negative trendline throughout the assessment.

FIGS. 5P through 5T illustrate a time series for the neurological attention metric. Attention includes a set of processes that enable the maintenance of goal-directed behavior in the face of distractions. This measure monitors the ability to maintain alertness and focus over time. In FIG. 5P, Patient 1's attention metric trend was consistent and steady during the 25-minute session. JD did not have difficulty maintaining alertness and focus over time. He wasn't distracted and completed all goals in the face of distractions without any indication of stress, effort, or concern. In FIG. 5Q, Patient 2's attention metric trend was consistent and steadily increased from 0.79 to over 1.3 over the 25-minute session. His initial attention metric was close to 1. His one-minute eyes attention metric closed baseline measuring Alpha Peak Frequency began at 0.95 at the beginning of the session and concluded at the peak of 1.70 in our live session. This number indicates how efficient and effective CM was in his ability to maintain and improve his alertness, focus over time and his ability to maintain goal directed behavior in the face of distractions throughout the session.

In FIG. 5R, Patient 3's attention metric trend was consistent and steadily increased from 0.59 to over 1.45 over the 25-minute session. His one-minute eyes closed attention metric baseline measuring Alpha Peak Frequency began at 0.8 at the beginning of the session and concluded at the peak of 1.25 in our live session. This number indicates how efficient and effective BB was in his ability to maintain and improve his alertness, focus over time and his ability to maintain goal directed behavior in the face of distractions throughout the session. BB's attention metric peaked at the beginning of the first imagery segment and again at the concluding imagery segment. In FIG. 5S, Patient 4's attention metric trend was consistent and barely increased over the 25-minute session. His one-minute eyes closed baseline attention metric measuring Alpha Peak Frequency began 1.4 at the beginning of the session and concluded under 1.2 in our live session. JC's ability to maintain and improve his alertness, focus over time and his ability to maintain goal directed behavior in the face of distractions throughout the session peaked at the beginning of each of the side-step hurdle motor task segments.

In FIG. 5T, Patient 5's attention metric trend was consistent throughout the 25-minute session. His one-minute eyes closed baseline attention metric measuring Alpha Peak Frequency began at 0.8 at the beginning of the session and concluded at a little over 1.2 in our live session. JM's attention metric peaked at the beginning of the second set of motor tasks at 2.95 and, 2.5 at the initial motor task, (i.e., unloaded squat and side-step hurdle). JM improved his alertness and focus while performing his motor tasks.

FIGS. 5U through 5Y illustrate a time series for the neurological workload metric. Workload is a measure that monitors the cognitive ability to interact with complex environments in a goal directed manner. Increases in workload are typically seen in tasks which place greater attentional, working memory, or other information processing demands on the neural system. In FIG. 5U, Patient 1's workload metric did not have much variation throughout the session. Workload which is the ability to efficiently interact with complex environments in a goal directed manner was implied in this trendline. JD operated with good energy and performed the exercises efficiently and with little extra effort or worry. His peak utilization of resource was the beginning of the motor tasks (ie., unloaded squat) and then trended downward throughout balance of session. In another words, the second set of motor tasks JD did not have to think about. He just performed all tasks as requested with little demand on workload.

In FIG. 5V, Patient 2 exhibited a consistent upward trend throughout the session. Workload which is the ability to efficiently interact with complex environments in a goal directed manner was apparent in this trendline. While CM used more resources initially performing the motor tasks, he utilized less information processing demands as evident by a decrease in workload metric during the second set of motor task activities. In FIG. 5W, Patient 3 exhibited a pattern consisting of an upward trend throughout the session. Workload which is the ability to efficiently interact with complex environments in a goal directed manner exhibited some apparent peaks and drop off in this trendline. BB used more resources for the R/L imagery at the beginning of the session and then for the single leg lunge and step over and then plateaued out with the drop off at the final imagery. BB used more information processing for the second motor task set of activities then the workload metric associated with the first set of motor task activities.

In FIG. 5X, Patient 4's uptrend was negligible throughout the session. Workload which is the ability to efficiently interact with complex environments in a goal directed manner was not apparent in this trendline. Most of the resources were used to perform both motor tasks which was evident by his tentative approach and fear in performing those motor tasks; and he utilized less information processing demands as evident by a decrease in workload metric during the second set of motor task activities. In FIG. 5Y, Patient 5 exhibited a slight upward trend throughout the session. Increased effort and workload which is the ability to efficiently interact with complex environments in a goal directed manner was apparent in this trendline. JM's workload began with the eyes closed baseline, decreased with imagery, and started to pick up with the initial set of motor tasks, specifically the unloaded squat. Workload decreased until the second set of motor tasks began. It is evident that JM utilized and used more resources initially performing the motor tasks.

FIG. 5Z illustrates sample extracts from assessment reporting provided by biometric interface 200 and/or server 112.

FIGS. 6A and 6B depict example neurological data assessments and visualizations that may be produced by the biometric interface. FIG. 6A depicts navigation between composite, time series displays within biometric interface 200. In the spectral power bands display interface, spectral power bands organize the EEG signal into discrete frequency bands, such as Delta (1-3 Hz), Theta (4-7 Hz), Alpha (8-12 Hz), Beta (13-30 Hz), and Gamma (30-100 Hz). In the EEG Theta display interface, Theta indicates the 4-7 Hz EEG frequency band associated with cognitive distraction or focus and attention. In the EEG Alpha display interface, Alpha indicates the 8-12 Hz EEG frequency band associated with cognitive readiness and alertness. In the EEG Beta 1 and Beta 2 display interface, Beta 1 indicates the 13-20 Hz EEG frequency band associated with cognitive planning. Beta 2 indicates the 20-30 Hz EEG frequency band associated with cognitive engagement. In the task workload metric, Task Load Metric measures the cognitive ability to interact with complex environments in a goal directed manner. Increases are typically seen in tasks which place greater attentional, working memory or other information processing demands on the neural system. In the Attention Metric display interface, the Attention Metric measures the set of processes that enable the maintenance of goal-directed behavior in the face of distractions. The Attention Metric may be used to monitor the ability to maintain alertness and focus over time.

FIG. 6B depicts navigation through views of a brain map 216, including a 3D brain volume view, a 3D brain axial view, and a 3D brain sagittal view, network connectivity, hemispherical power. For example, volume views use color to indicate regulation and dysregulation in regions of interest. These views also indicate hyper and hypo-coherence between regions, hemispheres and networks. Axial views indicate hemispherical disparities in regions of interest, and sagittal views indicate sub-surface activity. Related preprocessed EEG data is also shown.

FIG. 7 depicts example physiological data assessments and visualizations that may be produced by the biometric interface. The Heart Rate Variability (HRV) display interface measures the variation of time intervals between heartbeats, which has been shown to reflect emotional regulation, attentional regulation, and the ability to master breathing. HRV also provides insight into performance ability, and ability to maximize recovery. The Heart Rate display interface assists in training breathing patterns to match to HR patterns, thus reducing stress and anxiety while increasing focus and relaxation. Respiration data is used to train abdominal breathing for improved concentration, distraction control and stress response. The Trapezoidal EMG display interface indicates muscle tension and asymmetry between left and right sides. Data also indicates balance between heal and toe, proper body position, and ability to recover from stressors. Data can also be used to identify bracing, asses the likelihood of potential injury and determine readiness for return to play.

The EMG Imbalance display interface indicates whether the athlete is doing correct abdominal breathing or shallow/thoracic breathing. EMG Imbalance also indicates whether the non-dominate side is over and under reacting. The Galvanic Skin Response (GSR) display interface identifies the optimal state of emotional arousal levels. GSR also indicates fatigue, stress levels, focus, attention, over-thinking, and stress anticipation. The Peripheral Temperature display interface indicates the ability to learn to self-regulate stress responses. As temperature increases and more blood flows throughout the body and brain, the nervous system begins to calm down. Decreasing temperature indicates holding one's breath and an increased stress response.

FIG. 8 is a conceptual system diagram illustrating an example software system 400 for physiological and neurological rehabilitation of an athlete, according to aspects of the subject technology. The database 110 of the subject technology is configured to capture assessment data, function of training routines, and continual maintenance of aggregated protocols. According to various implementations, the database 110 is structured to record and maintain athlete information, including:
Profile data (excluding mental health) retrieved from existing/external records (e.g. Epic EHR) and collected during the system registration process
Baseline data collected during the initial neuro/physical assessment process.
Supplemental baseline data collected by from certain elite athletes
Sessions, segments, protocols, thresholds, results and prescriptives recorded as part of a injury prevention training routine
Post-injury/pre-rehabilitation baseline data
Data recorded as part of a injury rehabilitation training routine
Data recorded as part of a RTP (return-to-play) clearance process

The above athlete data may be supplemented by session playback recordings and related HTML reports, and may also be aggregated to form a separate, composite database from which actionable information may be drawn and protocols formulated.

Training data may be developed over time based on quantitative analytics of rehabilitation put in place by trained physical therapists, neurologists and other doctors. As therapies are validated for particular neuro-metrics and brain networks and weighted according to their efficacy for reestablishing baselines within those networks, and stored in a central database 110. The data is then accessible through a cloud based server 112 to facilitate selection of rehabilitation protocols for less trained practitioners (e.g., physical therapist) in remote locations. The quality of the guidance and precision grows as different types of participants are enlisted and the cohort expands over time.

Neurological training data may be obtained for a non-injured patient population. The training data may pertain to athletic performance reaction to various predetermined visual stimulation (including the foregoing tasks, activities, etc.). The training data may include measurements pertaining to the various neurological metric (activation, connectivity, asymmetry) for each measured brain region in a stable state, and baselines established for each metric/region/etc. These baselines may then be stored in the database 110 in a configuration that may be indexed by injury type, patient demographic, physiological information and/or other patient characteristics and the like. Likewise, the database 110 may include visual stimulations that are predetermined for rehabilitation of the various neurological metrics and/or networks, etc.

FIG. 9 is a conceptual diagram illustrating an example electronic system 500 for physiological and neurological rehabilitation of an athlete, according to aspects of the subject technology. Electronic system 300 may be a computing device for execution of software associated with one or more portions or steps of process 500, or components and processes provided by FIGS. 1, 2A, 2B, 2C, 3A, 3B, and 4-8. Electronic system 500 may be representative, in combination with the disclosure regarding FIGS. 1, 2A, 2B, 2C, 3A, 3B, and 4-8, of clinician device(s) 106 and/or the centralized server 112 described above. In this regard, electronic system 500 or computing device may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

An augmented reality device 500 may include various hardware devices, such as a head-mounted display that places images of both the physical world and virtual objects over the user's field of view, eyeglasses that employ cameras to intercept the real world view and re-display an augmented view through the eyeglass, a heads-up display, contact lenses that contain elements for display embedded in to the lens, virtual retinal display in which a display is scanned directly onto the retina or a user, an eye tap, that intercepts light and augments light that passes through the center of the lens of the eye of the wearer, and other similar devices. The sensors may be configured to map a position of a user object using x, y and z axis coordinates so as to identify a location of the user with respect to one or more objects in a particular environment.

Electronic system 500 may include various types of computer readable media and interfaces for various other types of computer readable media. In the depicted example, electronic system 500 includes a bus 508, processing unit(s) 512, a system memory 504, a read-only memory (ROM) 510, a permanent storage device 502, an input device interface 514, an output device interface 506, and one or more network interfaces 516. In some implementations, electronic system 500 may include or be integrated with other computing devices or circuitry for operation of the various components and processes previously described.

Bus 508 collectively represents all system, peripheral, and chipset buses that communicatively connect the numerous internal devices of electronic system 500. For instance, bus 508 communicatively connects processing unit(s) 512 with ROM 510, system memory 504, and permanent storage device 502.

From these various memory units, processing unit(s) 512 retrieves instructions to execute and data to process in order to execute the processes of the subject disclosure. The processing unit(s) can be a single processor or a multi-core processor in different implementations.

ROM 510 stores static data and instructions that are needed by processing unit(s) 512 and other modules of the electronic system. Permanent storage device 502, on the other hand, is a read-and-write memory device. This device is a non-volatile memory unit that stores instructions and data even when electronic system 500 is off. Some implementations of the subject disclosure use a mass-storage device (such as a magnetic or optical disk and its corresponding disk drive) as permanent storage device 502.

Other implementations use a removable storage device (such as a floppy disk, flash drive, and its corresponding disk drive) as permanent storage device 502. Like permanent storage device 502, system memory 504 is a read-and-write memory device. However, unlike storage device 502, system memory 504 is a volatile read-and-write memory, such a random access memory. System memory 504 stores some of the instructions and data that the processor needs at runtime. In some implementations, the processes of the subject disclosure are stored in system memory 504, permanent storage device 502, and/or ROM 510. From these various memory units, processing unit(s) 512 retrieves instructions to execute and data to process in order to execute the processes of some implementations.

Bus 508 also connects to input and output device interfaces 514 and 506. Input device interface 514 enables the user to communicate information and select commands to the electronic system. Input devices used with input device interface 514 include, e.g., alphanumeric key boards and pointing devices (also called “cursor control devices”). Output device interfaces 506 enables, e.g., the display of images generated by the electronic system 500. Output devices used with output device interface 506 include, e.g., printers and display devices, such as cathode ray tubes (CRT) or liquid crystal displays (LCD). Some implementations include devices such as a touchscreen that functions as both input and output devices.

Also, as shown in FIG. 9, bus 508 also couples electronic system 500 to a network (not shown) through network interfaces 516. Network interfaces 516 may include, e.g., a wireless access point (e.g., Bluetooth or WiFi) or radio circuitry for connecting to a wireless access point. Network interfaces 516 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 500 can be used in conjunction with the subject disclosure.

These functions described above can be implemented in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray R discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer,” “server,” “processor,” and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification.

Clause 1. A method comprising: receiving an injury type pertaining to a physical injury experienced by a patient; selecting, based on an injury type, one or more target regions of the patient's brain for assessment of a neurological metric associated with a neurological network of the patient's brain; providing one or more neurological sensors for measuring neurological activity within the one or more target regions of the patient's brain; providing, via a display screen, a first predetermined visual stimulation; initiating an athletic performance of the patient based on the first predetermined visual stimulation; obtaining, via one or more neurological sensors, first neurological data pertaining to the athletic performance of the patient reacting to the first predetermined visual stimulation, wherein the first neurological data comprises measurements pertaining to the neurological metric; determine a baseline data threshold for the neurological metric; comparing the obtained first neurological data to the baseline data threshold; selecting a second predetermined visual stimulation configured to prompt the neurological metric toward the baseline data threshold; obtaining, via the one or more neurological sensors, during the second predetermined visual stimulation, second neurological data pertaining to one or more actions of the patient responsive to the second predetermined visual stimulation: determining a deviation between the baseline data threshold and the second neurological data; determining, based on the deviation, a state of the neurological metric indicating whether the neurological metric adjusted toward the baseline threshold; and providing, on a display screen, an indication of the state of the neurological metric and a graphical representation of the second neurological data and the deviation.

Clause 2. The method of Clause 1, wherein the neurological network comprises a Mirror neuron Network, Salience Network, or Default Network of the patient's brain, the method further comprising: rendering electrical activity occurring in different sections of the brain during the one or more actions, wherein the graphical representation is based on the rendered electrical activity.

Clause 3. The method of Clause 1 or Clause 2, further comprising: generating a first task segment associated with the one or more actions, the first task segment comprising one or more physical or mental tasks; wherein the second visual stimulation is based on the first task segment and comprises visual instruction for prompting the person to perform the one or more physical or mental tasks, wherein the second neurological data is obtained in real-time responsive to a performance of at least one physical or mental tasks by the person.

Clause 4. The method of any one of Clauses 1 through 3, further comprising: obtaining physiological data during the second predetermined visual stimulation, wherein the physiological data comprises a heart rate, hear rate variability, respiration rate, ECG data, EKG data, EMG data, galvanic skin response, or peripheral temperature.

Clause 5. The method of any one of Clauses 1 through 4, wherein the neurological data comprises: spectral power bands, absolute or relative EEG power, neurological connectivity, EEG delta data, EEG theta data, EEG alpha data, EEG SMR data, EEG beta data, task load metrics, or attention metrics.

Clause 6. The method of any one of Clauses 1 through 5, wherein obtaining first or second neurological data comprises: measuring a stress response level and an arousal level in anticipation of a visual stimuli; and measuring a stress response level and a second arousal level responsive to a stress stimuli, the stress stimuli includes an auditory or physical stimulus.

Clause 7. The method of any one of Clauses 1 through 6, wherein the baseline data threshold comprises a first reaction time to the second predetermined visual stimulation, wherein determining the deviation comprises: determining a magnitude of deviation between the first reaction time and a second reaction time to the selected visual stimulation.

Clause 8. The method of any one of Clauses 1 through 7, further comprising: obtaining neurological training data pertaining to the athletic performance of a non-injured patient population reacting to the first predetermined visual stimulation, wherein the neurological training data comprises measurements pertaining to the neurological metric in a stable state; and determining the baseline data threshold for the neurological metric based on the training data.

Clause 9. The method of Clause 8, further comprising: subjecting a plurality of visual stimulation to an patient population associated with a predetermined injury; and selecting the first predetermined visual stimulation from the plurality of visual stimulations based on a performance of the predetermined visual stimulation in adjusting the neurological metric toward the baseline threshold.

Clause 10. The method of any one of Clauses 1 through 9, wherein the neurological metric is indicative of connectivity, activation, or symmetry measured in the one or more target regions of the patient's brain, the method further comprising: determining a level of connectivity, activation, or symmetry based on the obtained first or second neurological data.

Clause 11. A system comprising: a processor; and a memory device containing instructions, which when executed by the processor cause the processor to: facilitate performance of a method according to any one of Clauses 1 through 10.

Clause 12. A non-transitory computer-readable medium comprising instructions, which when executed by a computing device, cause the computing device to perform a method according to any one of Clauses 1 through 10.

### FURTHER CONSIDERATIONS

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit this disclosure.

The term website, as used herein, may include any aspect of a website, including one or more web pages, one or more servers used to host or store web related content, etc. Accordingly, the term website may be used interchangeably with the terms web page and server. The predicate words “configured to,” “operable to,” and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

1. A method comprising:
receiving an injury type pertaining to a physical injury experienced by a patient;
selecting, based on an injury type, one or more target regions of the patient's brain for assessment of a neurological metric associated with a neurological network of the patient's brain;
providing one or more neurological sensors for measuring neurological activity within the one or more target regions of the patient's brain;
providing, via a display screen, a first predetermined visual stimulation;
initiating an athletic performance of the patient based on the first predetermined visual stimulation;
obtaining, via one or more neurological sensors, first neurological data pertaining to the athletic performance of the patient reacting to the first predetermined visual stimulation, wherein the first neurological data comprises measurements pertaining to the neurological metric;
determine a baseline data threshold for the neurological metric;
comparing the obtained first neurological data to the baseline data threshold;
selecting a second predetermined visual stimulation configured to prompt the neurological metric toward the baseline data threshold;
obtaining, via the one or more neurological sensors, during the second predetermined visual stimulation, second neurological data pertaining to one or more actions of the patient responsive to the second predetermined visual stimulation;
determining a deviation between the baseline data threshold and the second neurological data;
determining, based on the deviation, a state of the neurological metric indicating whether the neurological metric adjusted toward the baseline threshold; and
providing, on a display screen, an indication of the state of the neurological metric and a graphical representation of the second neurological data and the deviation.

2. The method of claim 1, wherein the neurological network comprises a mirror neuron network, salience network, or default network of the patient's brain, the method further comprising:
rendering electrical activity occurring in different sections of the brain during the one or more actions, wherein the graphical representation is based on the rendered electrical activity.

3. The method of claim 1, further comprising:
generating a first task segment associated with the one or more actions, the first task segment comprising one or more physical or mental tasks,
wherein the second visual stimulation is based on the first task segment and comprises visual instruction for prompting the person to perform the one or more physical or mental tasks, and
wherein the second neurological data is obtained in real-time responsive to a performance of at least one physical or mental tasks by the person.

4. The method of claim 1, further comprising:
obtaining physiological data during the second predetermined visual stimulation,
wherein the physiological data comprises a heart rate, hear rate variability, respiration rate, ECG data, EKG data, EMG data, galvanic skin response, or peripheral temperature.

5. The method of claim 1, wherein the neurological data comprises:
spectral power bands, absolute or relative EEG power, neurological connectivity, EEG delta data, EEG theta data, EEG alpha data, EEG SMR data, EEG beta data, task load metrics, or attention metrics.

6. The method of claim 1, wherein obtaining first or second neurological data comprises:
measuring a stress response level and an arousal level in anticipation of a visual stimuli; and
measuring a stress response level and a second arousal level responsive to a stress stimuli, the stress stimuli includes an auditory or physical stimulus.

7. The method of claim 1, wherein the baseline data threshold comprises a first reaction time to the second predetermined visual stimulation, wherein determining the deviation comprises:
determining a magnitude of deviation between the first reaction time and a second reaction time to the selected visual stimulation.

8. The method of claim 1, further comprising:
obtaining neurological training data pertaining to the athletic performance of a non-injured patient population reacting to the first predetermined visual stimulation, wherein the neurological training data comprises measurements pertaining to the neurological metric in a stable state; and
determining the baseline data threshold for the neurological metric based on the training data.

9. The method of claim 8, further comprising:
subjecting a plurality of visual stimulation to a patient population associated with a predetermined injury; and
selecting the first predetermined visual stimulation from the plurality of visual stimulations based on a performance of the predetermined visual stimulation in adjusting the neurological metric toward the baseline threshold.

10. The method of claim 1, wherein the neurological metric is indicative of connectivity, activation, or symmetry measured in the one or more target regions of the patient's brain, the method further comprising:
determining a level of connectivity, activation, or symmetry based on the obtained first or second neurological data.

11. A system comprising:
a processor; and
a memory device containing instructions, which when executed by the processor cause the processor to:
facilitate performance of a method according to claim 1.

12. A non-transitory computer-readable medium comprising instructions, which when executed by a computing device, cause the computing device to perform a method according to claim 1.
